(19) United States
(12) Patent Application Publication
(10) Pub. No.: US 2018/0028217 A1
(43) Pub. Date: Feb. 1, 2018

(54) PROBE FOR TISSUE TREATMENT WITH A TANDEM SNARE

(71) Applicant: Tandem Technologies Ltd., Misgav (IL)

(72) Inventor: Noam HASSIDOV, Moshav Bustan HaGalil (IL)

(21) Appl. No.: 15/546,012

(22) PCT Filed: Feb. 2, 2016
(86) PCT No.: PCT/IL2016/050111

§ 371 Date: Jul. 25, 2017

Related U.S. Application Data
(60) Provisional application No. 62/274,806, filed on Jan. 5, 2016.
(60) Provisional application No. 62/110,627, filed on Feb. 2, 2015.

(51) Int. Cl. A61B 17/3205; A61B 1/31; A61B 17/00; A61B 17/295

(52) U.S. Cl. CPC A61B 17/32056; A61B 17/295; A61B 1/31; A61B 17/00234; A61B 2017/00269; A61B 2017/00358; A61B 2017/00353

(57) ABSTRACT

A device for cutting polyps via an endoscope lumen, including an over tube arranged to pass through an endoscope, the over tube including (1) a grasping tool arranged to be controlled from a proximal end of the endoscope and to controllably extend from a distal end of the endoscope, and (2) a cutting tool arranged to pass through the endoscope, the cutting tool arranged to be controlled from the proximal end of the endoscope and to controllably extend from the distal end of the endoscope, in which at least one of the grasping tool and the cutting tool is configured to bend away from the other one by including a pre-bent element configured to bend away when extended from the endoscope, and biased to a flat configuration when inside the endoscope, and the cutting tool is coupled to the grasping tool. Related apparatus and methods are also described.

### RELATED APPLICATION/S

This application is a continuation-in-part of U.S. Provisional Patent Application No. 62/274,806 filed 5 Jan. 2016, which is a continuation-in-part of U.S. Provisional Patent Application No. 62/110,627 filed 2 Feb. 2015.

The contents of all of the above applications are incorporated by reference as if fully set forth herein.

### FIELD AND BACKGROUND OF THE INVENTION

The present invention, in some embodiments thereof, relates to medical devices and methods, and, more particularly, but not exclusively, to devices and methods for removing polyps and/or undesirable tissue, and, even more particularly, but not exclusively, to devices and methods for removing polyps and/or undesirable tissue through an endoscope.

Additional background art includes:

U.S. Pat. No. 6,228,083 to Lands et al;

U.S. Patent Application Publication number 2013/0006234 of Couvillon Jr.;

U.S. Pat. No. 5,814,052 to Nakao et al;

U.S. Pat. No. 5,746,747 to McKeating; and

U.S. Pat. No. 5,630,813 to Kieturakis.

The disclosures of all references mentioned above and throughout the present specification, as well as the disclosures of all references mentioned in those references, are hereby incorporated herein by reference.

### SUMMARY OF THE INVENTION

An aspect of some embodiments of the disclosure includes apparatus and a method for inserting a grasping tool and a cutting tool through an endoscope's working channel, grasping a polyp, using the grasping tool to pull the polyp away from its base, sliding a cutting tool along the grasping tool and onto the polyp, and cutting the polyp. In some embodiments, since the grasping tool is holding onto the polyp, the polyp is easily extracted from a patient's body by virtue of being still grasped by the grasping tool after having been detached from the patient's body cavity.

In some embodiments, when the grasping tool is holding onto a polyp, the cutting tool can be moved relative to the grasping tool, enabling precise placement of the cutting tool on the polyp.

According to an aspect of some embodiments of the present invention there is provided a device for cutting polyps via an endoscope lumen, including an over tube arranged to pass through an endoscope, the over tube including (1) a grasping tool arranged to be controlled from a proximal end of the endoscope and to controllably extend from a distal end of the endoscope, and (2) a cutting tool arranged to pass through the endoscope, the cutting tool arranged to be controlled from the proximal end of the endoscope and to controllably extend from the distal end of the endoscope, in which at least one of the grasping tool and the cutting tool is configured to bend away from the other one by including a pre-bent element configured to bend away when extended from the endoscope, and biased to a flat configuration when inside the endoscope, and the cutting tool is coupled to the grasping tool.

According to some embodiments of the invention, the cutting tool is coupled to the grasping tool while the grasping tool and the cutting tool are inside the over tube, and the cutting tool is arranged to slide along the grasping tool when pushed toward a distal end of the grasping tool.

According to some embodiments of the invention, the cutting tool includes a loop coupled to the grasping tool while the grasping tool and the loop are inside the over tube, and the loop is arranged to slide over the grasping tool when pushed toward a distal end of the grasping tool.

According to some embodiments of the invention, the device further includes a grasping tool sheath enveloping the grasping tool, the cutting tool envelopes the grasping tool sheath while the grasping tool and the cutting tool are inside the over tube, and the cutting tool is arranged to slide along the grasping tool sheath and the grasping tool when pushed toward a distal end of the grasping tool.

According to some embodiments of the invention, further including a tensioning mechanism attached to the grasping tool, operational to maintain grasping force on tissue by maintaining tension between a distal end of the grasping tool and a manipulating mechanism at a proximal end of the grasping tool.

According to some embodiments of the invention, the tensioning mechanism includes a spring. According to some embodiments of the invention, the tensioning mechanism further includes a locking mechanism.

According to some embodiments of the invention, further including a tensioning mechanism attached to the cutting tool, operational to maintain cutting force on tissue by maintaining tension between a distal end of the cutting tool and a manipulating mechanism at a proximal end of the cutting tool.

According to some embodiments of the invention, the tensioning mechanism includes a spring. According to some embodiments of the invention, the tensioning mechanism further includes a locking mechanism.

According to some embodiments of the invention, the device further includes a cutting tool sheath enveloping a portion of the cutting tool, and the cutting tool is configured to controllable extend from the cutting tool sheath.

According to some embodiments of the invention, the over tube is sized and shaped to be inserted into a body through a lumen of the endoscope.

According to some embodiments of the invention, the over tube includes a separator between a first lumen through the over tube enveloping the grasping tool and a second lumen through the over tube enveloping the cutting tool.

According to some embodiments of the invention, the endoscope is one of a group consisting of an arthroscope, an amnioscope, a laryngoscope, a colposcope, a hysteroscope, a laparoscope, a sygmoidoscope, a hysteroscope, a gastroscope, a colonoscope, and a cystoscope.

According to some embodiments of the invention, an outer diameter of the endoscope is less than 14 mm.

According to some embodiments of the invention, the grasping tool sheath is configured to bend away from the cutting tool when the cutting tool is extended beyond the grasping tool and the grasping tool sheath is extended out of the over tube.

According to some embodiments of the invention, the grasping tool sheath further includes at least one shape memory insert configured to bend away from the cutting tool when the grasping tool sheath is extended out of the over tube.

According to some embodiments of the invention, the at least one shape memory insert extends from a distal end of the grasping tool sheath to a proximal end of the over tube.

According to some embodiments of the invention, the grasping tool sheath further includes at least a spring insert configured to bend away from the cutting tool when the grasping tool sheath is extended out of the endoscope.

According to some embodiments of the invention, the cutting tool includes a snare. According to some embodiments of the invention, the cutting tool includes a loop. According to some embodiments of the invention, the cutting tool includes an electrically conductive wire.

According to some embodiments of the invention, the grasping tool is one of a group consisting of a loop, a snare, a grasper, a biopsy forceps, a tweezers-like grasper, and a harpoon.

According to an aspect of some embodiments of the present invention there is provided a device for excising polyps via an endoscope lumen, the device including a grasping tool arranged to pass through a lumen of an endoscope, the grasping tool arranged to be controlled from a proximal end of the endoscope and to controllably extend from a distal end of the endoscope, and a cutting tool arranged to pass through the lumen of the endoscope, the cutting tool arranged to be controlled from the proximal end of the endoscope and to controllably extend from the distal end of the endoscope, in which at least one of the grasping tool and the cutting tool is configured to bend away from another one of the grasping tool and the cutting tool when extended from the endoscope.

According to an aspect of some embodiments of the present invention there is provided a device for grasping polyps via an endoscope lumen, the device including a grasping tool arranged to pass through the lumen of the endoscope, a tensioning mechanism attached to the grasping tool and to a manipulating control configured to be at a proximal end of the endoscope, in which the tensioning mechanism is configured to maintain grasping tension on a polyp when the endoscope is flexed.

According to some embodiments of the invention, the tensioning mechanism further includes a lock for maintaining tension following locking without requiring tension on the manipulating control.

According to an aspect of some embodiments of the present invention there is provided a device for cutting polyps via an endoscope lumen, the device including a cutting tool arranged to pass through the lumen of the endoscope, a tensioning mechanism attached to the cutting tool and to a manipulating control configured to be at a proximal end of the endoscope, in which the tensioning mechanism is configured to maintain cutting pressure on a polyp during cutting without requiring tension on the manipulating control.

According to some embodiments of the invention, the tensioning mechanism further includes a lock for producing tension before locking, and applying the tension by unlocking.

According to an aspect of some embodiments of the present invention there is provided a surgical method including extending a grasping tool from a distal end of an endoscope, grasping a portion of a polyp with the grasping tool, extending the grasping tool further from the distal end of the endoscope, thereby causing the grasping tool to bend away from a base of the polyp, extending a cutting tool coupled to the grasping tool from the distal end of the endoscope such that the cutting tool slides along the grasping tool, beyond a distal end of the grasping tool and onto the polyp, snaring the polyp with the cutting tool, and cutting the polyp using the cutting tool.

According to some embodiments of the invention, the cutting includes electrically cutting.

According to some embodiments of the invention, the extending the grasping tool further from the distal end of the endoscope causing the grasping tool to bend away from a base of the polyp includes extending a sheath enveloping the grasping tool further from the distal end of the endoscope causing the grasping tool sheath to bend away from a base of the polyp.

According to some embodiments of the invention, further including using a camera at a distal end of the endoscope to observe the grasping the portion of a polyp with the grasping tool, and following the extending the grasping tool further from the distal end of the endoscope causing the grasping tool to bend away from a base of the polyp, using the camera to observe the snaring the polyp with the cutting tool.

According to some embodiments of the invention, further including using the camera to observe the cutting the polyp using the cutting tool.

According to some embodiments of the invention, further including retrieving the cut polyp from a patient's body using the grasping tool. According to some embodiments of the invention, further including pulling the cut polyp into the endoscope.

According to some embodiments of the invention, the cutting tool envelopes the grasping tool while the grasping tool and the cutting tool are inside the endoscope, and the cutting tool is arranged to slide over the grasping tool when pushed toward a distal end of the grasping tool.

According to an aspect of some embodiments of the present invention there is provided a surgical method including extending a first tool from a distal end of an endoscope, grasping a portion of a polyp with the first tool, causing the first tool to bend away from a base of the polyp, extending a second tool coupled to the first tool from the distal end of the endoscope such that the second tool slides along the first tool, beyond a distal end of the first tool and onto the polyp, snaring the polyp with the second tool, and cutting off at least a portion of the polyp using the second tool.

According to some embodiments of the invention, the cutting includes electrically cutting.

According to some embodiments of the invention, further including pulling the cut off portion of the polyp out of a patient's body. According to some embodiments of the invention, further including pulling the cut off portion of the polyp into the endoscope.

According to an aspect of some embodiments of the present invention there is provided a surgical method including extending a grasping tool from a distal end of an endoscope, grasping a portion of a polyp with the grasping tool, extending the grasping tool further from the distal end of the endoscope, thereby causing the grasping tool to bend away from a base of the polyp, extending a cutting tool coupled to the grasping tool from the distal end of the endoscope such that the cutting tool slides along the grasping tool, beyond a distal end of the grasping tool and onto the polyp, snaring the polyp with the cutting tool, cutting the polyp using the cutting tool, and retrieving the cut polyp from a patient's body using the grasping tool.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are simplified schematic drawings of a prior art endoscopic setting used to treat a target tissue in a monopolar or a bipolar manner;

FIGS. 2A and 2B are simplified schematic drawings of a prior art use of a bipolar probe;

FIGS. 2C-2J are simplified schematic drawings of a prior art use of a monopolar probe;

FIG. 2K is a simplified schematic drawing of current flow within tissue of a body according to prior art;

FIGS. 3A-3D are simplified schematic drawings of a probe according to an example embodiment of the invention;

FIGS. 4A-4C are simplified schematic illustrations of a probe according to some example embodiments of the present invention;

FIGS. 4D-4E and FIGS. 5A-5C are simplified schematic illustrations of a probe according to some embodiments of the present invention;

FIGS. 5D-5E are simplified schematic illustrations of a probe according to some example embodiments of the present invention;

FIGS. 6A-6B are simplified schematic illustrations of a probe according to some example embodiments of the present invention;

FIG. 6C is a simplified schematic illustration of a probe according to an example embodiment of the invention;

FIG. 6D is a simplified schematic illustration of a probe according to an example embodiment of the invention;

FIGS. 7A-7H are simplified schematic illustrations of a probe according to some example embodiments of the invention;

FIGS. 8A-8F are simplified schematic illustrations of a probe according to some example embodiments of the invention;

FIGS. 9A-9D are simplified schematic illustrations of a probe according to some example embodiments of the invention;

FIGS. 10A-10B are simplified schematic illustrations of a probe according to some example embodiments of the invention;

FIGS. 10C-10J are simplified schematic illustrations of a probe with a multi lumen pipe according to some example embodiments of the invention;

FIGS. 11A1-3 and 11B are simplified schematic illustrations of a probe according to some example embodiments of the invention;

FIGS. 11C-11N are simplified schematic illustrations of a probe according to some example embodiments of the invention;

FIGS. 12A-12C and 13A-13G are simplified schematic illustrations of a probe according to some example embodiments of the invention;

FIGS. 14A-14F are simplified schematic illustrations of a probe according to some embodiments of the invention;

FIGS. 15A-15D, 16A-16D and 17A-17F are simplified schematic illustrations of a probe according to some embodiments of the invention;

FIGS. 18A-18C are simplified schematic illustrations of a snare and a polyp according to prior art;

FIGS. 19A-19F are simplified schematic illustrations of a probe according to an embodiment of the invention;

FIGS. 20A-20B are simplified schematic illustrations of a probe with a protective cover according to an embodiment of the invention;

FIG. 21 is a simplified flow chart of a method for removing a polyp according to an example embodiment of the invention;

FIGS. 22A-22D are simplified schematic illustrations of a probe according to an example embodiment of the invention;

FIG. 23 is a simplified flow chart of a method for removing a polyp according to an example embodiment of the invention; and

FIG. 24 is a simplified flow chart of a method for removing a polyp according to an example embodiment of the invention.

### DESCRIPTION OF SPECIFIC EMBODIMENTS OF THE INVENTION

The present invention, in some embodiments thereof, relates to medical devices and methods, and, more particularly, but not exclusively, to devices and methods for removing polyps and/or undesirable tissue, and, even more particularly, but not exclusively, to devices and methods for removing polyps and/or undesirable tissue through an endoscope.

An endoscope can include:

a rigid or flexible tube;

a light delivery system to illuminate an organ or an object under inspection. A light source may be outside the body and light directed via an optical fiber system or a LED light source may be used inside the body;

a lens system transmitting the image from an objective lens to a viewer, typically a relay lens system in the case of rigid endoscopes or a bundle of fiber optics in the case of a fiberscope;

an eyepiece—modern instruments may be video scopes, with no eyepiece. A camera may transmit an image to a screen for observation or image capture; and

one or more additional channels or lumens to enable entry of medical instruments or manipulators.

Endoscopes are often flexible, so mechanical control of medical instruments or manipulators from a proximal end of the endoscope to a distal end of the endoscope is limited. Mechanical control of manipulators along a push-pull axis is possible, by pushing or pulling one end of a manipulator extending along a lumen of the endoscope. Control for rotating a manipulator is possible by rotating a manipulator, although a flexible manipulator may degrade the control, making rotating a distal end of a manipulator somewhat unpredictable when rotating a proximal end of the manipulator. In contrast, some rigid laparoscopes are built to enable additional mechanical control for side to side and up and down on the distal end.

An aspect of some embodiments of the invention includes a tool for grasping a polyp, through a lumen of an endoscope, by pushing and/or pulling at a proximal end of the endoscope. The grasping tool is also controlled to shift laterally, relative to the endoscope axis by pushing and/or pulling at the proximal end of the endoscope. Such capability is taught herein, in some embodiments, for grasping a polyp and also pulling the polyp away from its base, or away from a lumen wall, if need be.

In some embodiments, the grasping tool includes a tensioning mechanism for maintaining grasping force of the grasping tool on a polyp during possible shifting of the endoscope or patient body. In some embodiments, the tensioning mechanism includes a lock such that the tensioning mechanism may be applied and the tension remains locked until a physician chooses to unlock. In some embodiments, the tensioning mechanism includes a spring along a wire between the grasping tool and a manipulating handle of the grasping tool.

In some embodiments, a cutting tool is coupled to the grasping tool, and caused to slide along the grasping tool, thereby guiding the cutting tool onto the grasped polyp.

In some embodiments, the cutting tool is shaped such that after sliding off an end of the grasping tool, the cutting tool encircles some or all of the polyp, while the grasping tool is still holding the polyp.

In some embodiments, the two tools are caused to shift laterally away from each other, so that the grasping tool pulls the polyp and the cutting tool slides down the polyp toward a base of the polyp.

In some embodiments, the endoscope includes an imaging system at its distal end. The shifting of the polyp laterally by the grasping tool potentially moves the grasping tool and a top of the polyp away from a center of the field of view (FOV) of an imager, and enables the imager to image the cutting tool and a middle or base of the polyp.

In some embodiments, the cutting tool includes a tensioning mechanism for maintaining cutting pressure of the cutting tool on a polyp, during possible change of dimensions of the cutting area, shifting of the endoscope or patient body. In some embodiments, the tensioning mechanism includes a lock such that the tensioning mechanism may be tensioned prior to cutting, and the tensioning unleashed to act on the cutting area when the cutting is to begin, maintaining the tension during cutting. In some embodiments, the tensioning mechanism includes a spring along a wire between the cutting tool and a manipulating handle of the cutting tool.

An aspect of some embodiments of the invention includes apparatus and methods for grasping with a grasping tool, through a lumen of an endoscope or an add-on lumen coupled to the endoscope at a distal end of the endoscope, by pushing and/or pulling at a proximal end of the endoscope, yet causing the grasping tool to shift laterally in addition to longitudinally. Such capability is taught herein, in some embodiments, for grasping a polyp and pulling the polyp away from its base.

In some embodiments, causing the grasping tool to shift laterally is optionally done by having at least a portion of the grasping tool be configured such that when the grasping tool is within the endoscope, or within a sheath or an over sheath, the grasping tool is constrained to be relatively straight, and when the grasping tool is extended beyond the endoscope/sheath/over sheath, the grasping tool reverts to bending, in a desired direction such as away from a longitudinal axis of the endoscope. In order to achieve the bending, in some embodiments the grasping tool or a portion thereof may be formed of a spring which is pre-bent, or of a shape memory material such as Nitinol, which is pre-bent. In some embodiments, a sheath or a portion thereof enveloping the grasping tool may be produced of a material and be pre-bent as described above. In some embodiments, an additional insert or spring may be produced of a material and be pre-bent as described above, and be placed in a sheath enveloping the grasping tool.

An aspect of some embodiments of the invention includes having a cutting tool coupled to a grasping tool yet free to slide along the grasping tool, so that the cutting tool can be slid off the grasping tool onto a polyp without a need to observe the cutting tool and/or maneuver the cutting tool.

In some embodiments the cutting tool may be, by way of some non-limiting example, a 360° loop looped over the grasping tool, or be shaped as a U shape or L shape engaged with the grasping tool.

An aspect of some embodiments of the invention includes having the grasping tool grasp while in a field of view (FOV) of the endoscope, then pull the polyp aside or up to clear a center of the FOV of the grasping tool and grasped portion of the polyp, and bring a base of the polyp into the field of view. In some embodiments the grasping tool is moved aside or up so as not to obstruct the field of view of the endoscope, or to lessen obstruction of the field of view of the endoscope by the grasping tool and/or the polyp.

An aspect of some embodiments of the invention includes extending the cutting tool into the field of view after the grasping by the grasping tool, and in some embodiments even after the pulling aside. The cutting tool does not appear in the field of view and does not interfere with the view during the grasping, and does appear when it has to be positioned for the cutting.

An aspect of some embodiments of the invention includes performing the cutting by the cutting tool within the field of view of the endoscope, without the grasping tool obstructing part of the field of view.

An aspect of some embodiments of the invention includes causing the cutting tool to shift laterally in addition to longitudinally relative to a polyp and/or relative to a polyp grasped by the grasping tool. Such capability is taught herein, in some embodiments, for optionally shifting the cutting tool away from a companion grasping tool. Shifting the cutting tool away from the grasping tool can assist in shifting the cutting tool toward a base of the polyp, which is often desirable. In some embodiments, the cutting tool slides onto the polyp over the grasping tool which is grasping the head of the polyp, and when the cutting tool shifts away from the grasping tool, the cutting tool shifts along the polyp toward the base of the polyp.

In some embodiments, causing the cutting tool to shift is optionally done by having at least a portion of the cutting tool be configured such that when the cutting tool is within the endoscope, or within a sheath or an over sheath, the cutting tool is constrained to be relatively straight, and when the cutting tool is extended beyond the endoscope/sheath/over sheath, the cutting tool reverts to bending, in a desired direction such as away from a longitudinal axis of the endoscope. In order to achieve the bending, in some embodiments the cutting tool or a portion thereof may be formed of a spring which is pre-bent, or of a shape memory material such as Nitinol, which is pre-bent. In some embodiments, a sheath or a portion thereof enveloping the cutting tool may be produced of a material and be pre-bent as described above. In some embodiments, an additional insert or spring may be produced of a material and be pre-bent as described above, and be placed in a sheath enveloping the cutting tool.

In some embodiments, a spreading element is included between the grasping tool and the cutting tool, which is controlled from the proximal end of the endoscope for pushing aside the grasping tool or the cutting tool or both.

The endoscope potentially acts as a platform from which the grasping tool and the cutting tool extend.

In some embodiments, the endoscope position optionally serves as a base or anchor off of which the grasping tool and/or the cutting tool push off laterally, the endoscope potentially being a larger and more stable mechanical element than the grasping or the cutting tool.

In some embodiments, causing the grasping tool to shift laterally is optionally done by (a) having at least a portion of the grasping tool protruding from the endoscope's working channel and: (b) at least a portion of the grasping tool's shaft enfolded within endoscope's working channel in such a manner that the shaft is free to move in a linear manner within such working channel and/or to rotate around its main axis; and/or (c) by a spring that is housed in one end (i) in the protruding portion and in the other end (ii) in the shaft enfolded within the working channel.

An aspect of some embodiments of the invention includes having a cut off portion of a polyp or other cut off body portion still grasped by the grasping tool after the cutting, so that the polyp does not need to be hunted and captured or re-captured following the cutting. Removal of the polyp from a patient's body is easily performed by the grasping tool, which has in such a case not left its grasp on the polyp during or after the cutting. Removal of a polyp is often an essential part of the surgical procedure, so as to be taken for pathological analysis.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Bipolar & Monopolar Diathermic Technology

Today, diathermic technology is divided, generally speaking, to two main types; monopolar and bipolar.

Monopolar current flow: During usage of a monopolar system the physician use a tool with one electrode to treat the target tissue wherein the second electrode is attached to the outer body's surface.

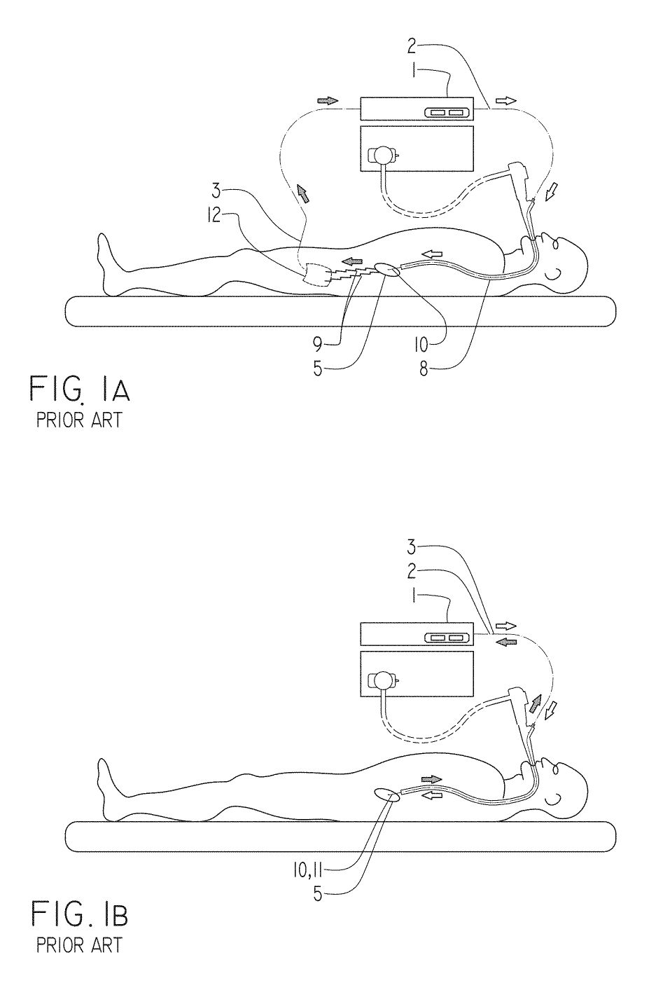

Reference is now made to FIGS. 1A and 1B, which are simplified schematic drawings of a prior art endoscopic setting used to treat a target tissue in a monopolar or a bipolar manner.

As rendered in FIG. 1A—The first electrode is known as the active electrode 10 may be hand held (not shown) during open surgery or inserted in to a body cavity via an introducer 8 (e.g. endoscope, laparoscope, colonoscope and other tools). Such a probe is designed to treat a given tissue 5 (e.g. a snare designed to remove a polyp within the colon using a colonoscope). A second electrode is known as a grounding pad 12 (also known as return, remote return or neutral electrode) will be attached to the body's skin using a special conductive glue.

A path of electrical current enfolds the generator 1, conductive cable 2, active electrode 10, target tissue 5 that should be treated, other tissues within the body 9, grounding pad 12, conductive cable 3 and the generator 1. The current travels in a path of least resistance through the patient's body and may harm not only the target tissue.

A neutral electrode or grounding pad should be placed as close as possible to a treatment site in order to keep the electrical circuit as short as possible (for example the grounding pad will be placed on the hip during colonoscopy or gastroscopy).

Bipolar current flow: During usage of a bipolar system the physician uses a tool with two electrodes enfolded in the tip of the tool to treat the target tissue. The tool may be hand held (not shown) or inserted via an introducer as described above.

As rendered in FIG. 1B—The path of the electrical current enfolds the generator 1, conductive cable 2, active electrode 10, target tissue that should be treaded 5, neutral electrode 11, conductive cable 3, and the generator 1. The current travels in only within the tissue that the tip of the probe comes in contacts with.

Generally, the power used in monopolar setting to treat a given tissue is higher than bipolar setting. Monopolar tools are considered as simpler and cheaper than bipolar tools.

The terms “endoscope” and “colonoscope” are used throughout the present specification and claims to mean an introducer, a device designed to introduce a probe in to a body's cavity.

For ease of use the term colonoscope will be used herein to describe an introducer. An introducer is a device designed to introduce a probe in to a body's cavity (e.g. Colonoscope, Gastroscope, Laparoscope, Laparoscopic port, Cystoscope, pipe and other devices with or without imaging capabilities, flexible or ridge devices, with or without steering capabilities).

For the ease of use the term colon will be used to describe any cavity of the body. Such a body cavity may be defined as a lumen within the body (e.g. colon, stomach, abdominal cavity during laparoscopic surgery, urinary bladder, sinus, lung and other cavities within the body). Such a body cavity may be defined as a confined or a partly confined lumen of the body (e.g. nasal structures, mouth, rectum, vaginal cavity and other cavities). Such a body cavity may be defined as cavity that may be accessed during treatment (e.g. during open surgery of the abdominal cavity).

The term “loop” is used throughout the present specification and claims to mean a snare or grasping tool, and in some non-limiting examples the grasping tool is indeed in a shape of a loop. However, when a tool such as a snare or a loop is used for grasping, it does not have to be in a shape of a loop, and may be in a shape of a grasper; a biopsy forceps; a tweezers-like grasper; a harpoon, and similar grasping tools.

The term “cutting tool” is used throughout the present specification and claims to mean a cutting tool, whether cutting mechanically or by electric current. In some non-limiting examples the cutting tool is in a shape of a loop or a snare; however the shape of a loop is not meant to be limiting for the cutting tool.

Examples of Probes and Their Problems

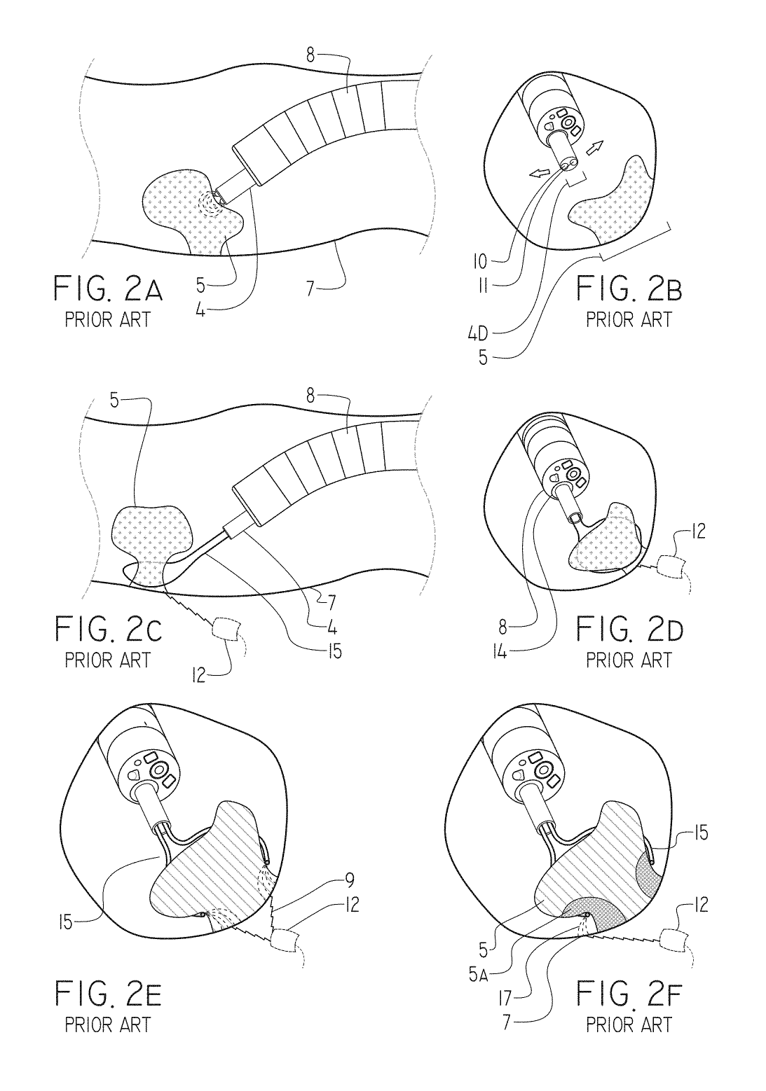

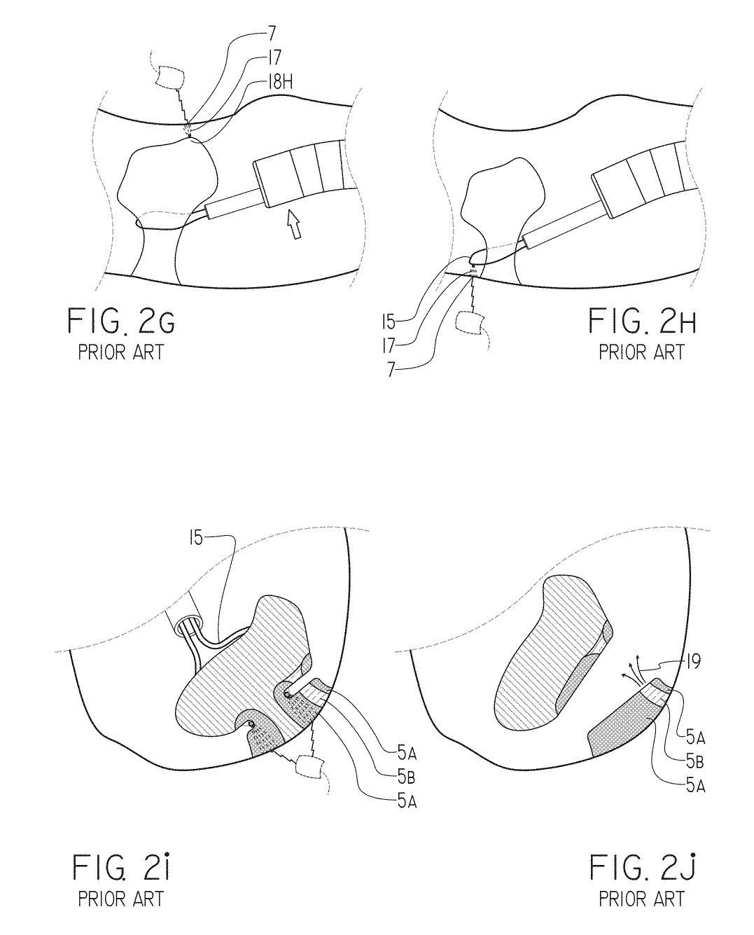

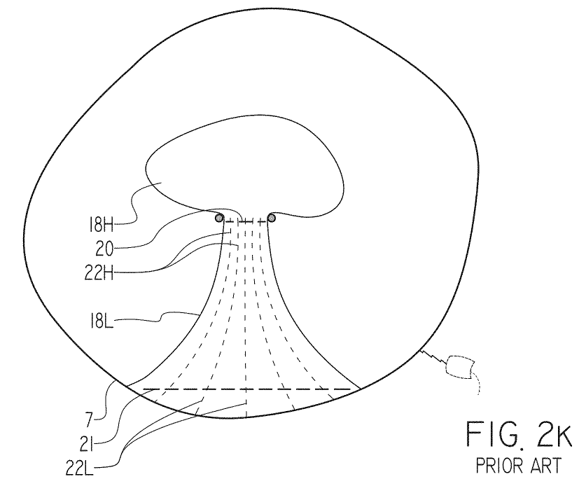

Reference is now made to FIGS. 2A and 2B, which are simplified schematic drawings of a prior art use of a bipolar probe.

As rendered in FIG. 2A-2B—One of the bipolar diathermic probes 4 is an elongated probe that can be inserted via a colonoscope 8 in to the colon 7. The probe tip size 4D is mostly smaller than the size of the treated tissue 5. Such a probe 4 enfolds two electrodes 10, 11 located at the tip of the probe 4. The physician places the probe's tip on the first part of the target tissue 5 and activates the generator; and a small part of the tissue is treated. The physician moves the tip around the target tissue to complete the treatment.

Such movement is simple to execute during open surgery, but complex to execute when a colonoscope is used (e.g. when a physician uses remote steering of the colonoscope to move the tip of the probe around the treated tissue or move the colonoscope back and forward). Such a probe may be inserted via a colonoscope working channel, and the probe's outer diameter is smaller than the colonoscope working channel inner diameter. For the ease of use such a probe will be named as Twin Tip Ball Based Probe or “Twin Tip BB Probe”.

Reference is now made to FIGS. 2C-2J, which are simplified schematic drawings of a prior art use of a monopolar probe.

As rendered in FIGS. 2C-2D—One of the monopolar diathermic probes is a polypectomy snare probe also called a snare loop or loop. The elongated probe 4 enfolds one electrode 15 with a round shape that can be opened or closed by the physician. The physician inserts the snare 4 via the colonoscope 8 working channel 14 and steers the snare's loop 15 in such a manner that it will be placed around the treated tissue 5, then closes the loop remotely. This task is relatively simple to master during open surgery but complex to execute as a remote steering of the colonoscope in the confined space of the colon. The closing force applies by the physician on the loop effects the efficacy of the treatment and incorrect force (too much or too little) may generate undesirable clinical results (e.g. bleeding or damage to a healthy tissue). Once the physician activates the generator the current will flow via the loop 15 acting as the active electrode, the tissue that should be treated 5, the colon wall 7 and other tissues of the body, the grounding pad 12, and back to the generator.

As rendered in FIG. 2E—Once the physician closes the snare's loop 15 with minimal closing force or applies no closing force on the loop during the treatment, the treated tissue in contact with the loop 15 (the active electrode) will potentially be coagulated due to flow of the current via the treated tissue, body's tissue 9 and the grounding pad 12.

As rendered in FIG. 2F—It is possible that during the treatment, the treated tissue 5 may be coagulated and dehydrated in such a manner that the tissue resistance to current flow will rise to high levels, and due to the minimal closing force on the loop, such treated tissue will be kept intact and act as an insulator tissue 5A. In such a monopolar scenario of high tissue resistance, the current may find a new path 17 to flow from the snare's loop 15 (active electrode) to the grounding pad 12 (neutral electrode) via any tissue with low resistance, for example via the colon's wall 7. Such undesirable current flow might generate tissue damage and even colon wall perforation. Due to such risk it is recommended that (i) the snare's loop be closed with a reasonable force and; (ii) after the closure of the snare on the polyp leg the physician should steer the snare's loop upwards and as far away from the colon wall as possible and; (iii) keep the head of the polyp far away from the upper wall of the colon.

As rendered in FIG. 2G—Such overdoing in upward steering may result in undesirable movement of the polyp head 18H in such a manner that the polyp head will touch the remote (or upper in this example) colon wall 7 resulting in a new and undesirable path 17 for the current to flow. Such undesirable flow path may generate colon wall damage and even colon perforation.

As rendered in FIG. 2H—Trying to generate current without steering up the snare's loop first may yield an undesirable flow path 17 between the snare's loop 15 distal part and the lower part of the colon 7 wall. Such undesirable flow path may generate colon wall damage and even colon perforation.

As rendered in FIGS. 2i-2J—If the physician closes the snare's loop using maximal or excessive closing force during the treatment. The tissue in contact with loop 15 (active electrode) may potentially be forced to (i) coagulate and dehydrate 5A in a proper manner or (ii) the tissue 5B will yield locally and tear due to the reduction of mechanical properties by the current flow and heating of the tissue. Once parts of the tissue become weaker the loop might slice through the tissue due to the excessive closing force. Some blood vessels within such tissue 5B might be left untreated and bleeding 19 might start. In such a monopolar scenario of excessive force applied to close the loop, some tissue might be treated 5B in a partial manner while other parts of the tissue might be treated properly 5A resulting in post procedure bleeding (that may start immediately after the removal of the snare from the tissue, within few minutes or within several hours after the snare was removed).

Reference is now made to FIG. 2K, which is a simplified schematic drawing of current flow within tissue of a body according to prior art.

As rendered in FIG. 2K, closing the loop on the polyp leg yields a small cross section within the tissue trapped in the loop, and more detailed within the plane of the loop. Such small cross section 20 yield high current density 22H thus tissue treatment will be applied in proximity to the loop and not in an undesirable locations (e.g. in near the colon wall 7). The big cross section 21 yield low current density 22L thus tissue will not be treated, or at least receive less of a treatment, in such a location.

The current density in a polyp leg, as described in a review by Curtiss, L. E. (1973) titled “High frequency currents in endoscopy: A review of principles and precautions”, published in Gastrointestinal Endoscopy, 20, 9-12, changes according to changes in a leg's diameter or the leg's cross section. For example, the cross section marked as 20 is located in the transition area between the polyp leg 18L and the polyp head 18H. In such a location the loop is being closed in this example and the outer diameter of the polyp leg is 3.5 mm yielding a ˜10 sq.mm cross section. The cross section marked as 21 is located in the transition area between the polyp leg 18L and the wall of the colon 7. In such location the outer diameter is 11 mm yielding a ˜100 sq.mm. Such a difference between the cross sections 20, 21 will generate high thermal activity in the first section 20 and much less thermal activity in the second section 21.

The physician is required to balance the force applied on the snare's loop with minimal feedback, since he can only speculate what is going on within the treated area. In such an undesirable user-dependant situation, closing the snare's loop as the process goes on, doing it with the right force, not too much, not too little while using a long snare that might have bends along the way, the results vary and bleeding happens from time to time.

Examples Diathermic Technology & the “Bring to Me Snare Concept”

Probes according to example embodiments of the invention will be described hereafter. Such probes may be used in an open surgery setting or in via an introducer within a lumen of the body. For ease of use the term working channel shall describe any lumen within such introducer enabling the passage of a probe in such a lumen of the body.

For the ease of use the term colon will be used to describe a cavity of the body. Such body's cavity may be defined as confined lumen within the body (e.g. colon, stomach, abdominal cavity during laparoscopic surgery, urinary bladder and cavities within the body). Such body's cavity may be defined as a partly confined lumen of the body (e.g. nasal structures, mouth, rectum, vaginal cavity and other cavities). Such body's cavity may be defined as cavity that may be accessed during treatment (e.g. during open surgery of the abdominal cavity).

For the ease of use the term snares or loops will be used to describe the probe and pedunculated polyp or polyp will be used to describe any type and shape of treated tissue (e.g. colonic polyps, sinus polyps, appendix, gall bladder, flat adenoma, skin mole and any other tissue or organ within a body's cavity or of the body in general).

As described above in FIG. 2B, the bipolar Twin Tip BB Probe touches only small areas or the treated tissue, and the physician is required to steer the tip of the probe in order to complete the treatment. Moreover the treatment is applied mostly to the outer surface of the tissue and cutting a polyp leg is hard to perform using such probe.

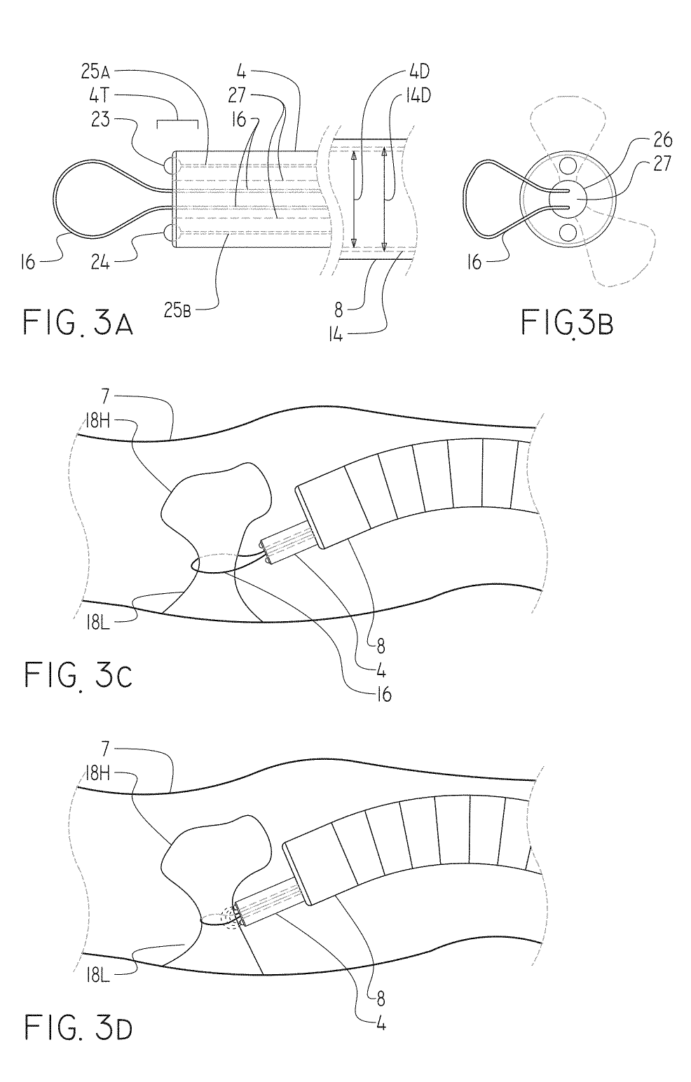

Reference is now made to FIGS. 3A-3D, which are simplified schematic drawings of a probe according to an example embodiment of the invention.

As rendered in FIGS. 3A-3B—A probe 4 is inserted in to the colon via the colonoscope 8 working channel 14. The probe's Outer Diameter 4D (OD) is smaller than the working channel Inner Diameter 14D (ID) enabling the passage of such probe via the working channel. The probe tip 4T enfolds two spherical electrodes 23, 24. The electrodes are connected via conductive wires 25A, 25B to the probe's handle (not shown) and to a generator. The tip of the probe enfolds an opening 26 enabling the introduction of a loop 16 from such opening in to the colon. The loop optionally spans from the tip of the probe via a lumen 27 within the probe and up to the probe's handle (not shown). The physician can move the probe's handle in such a manner that the loop will move forward and backwards within the lumen 27 yielding opening or closing motion within the tip of the loop, protruding from the tip of the probe. The loop can be completely retracted within the lumen if needed, for example during the insertion of the probe in to the colonoscope working channel.

As rendered in FIG. 3B—Once the tip of the probe protrudes from the working channel the physician may open or close the loop by manipulating the handle. The physician may also rotate the probe, rotate the loop within the probe lumen, move the probe in a linear manner within the working channel or steer the colonoscope tip to obtain optimal location of the probe's loop over the polyp.

As rendered in FIG. 3A, 3C-3D—The probe's loop 16 is optionally placed over the polyp head 18H and on the polyp leg 18L. Once the loop 16 is closed by the physician, the generator may be activated and the current flow from the generator (not shown) via the first conductive wire 25A, the first electrode 23, the treated tissue, the second electrode 24, the second conductive wire 25B and back to the generator. The loop 16 in this example may be made from a non-conductive material (but not necessarily be limited to such property). During the treatment the loop keeps the treated tissue in contact with the electrodes, for example by a force applied by the physician on the loop's handle. For the ease of use such a probe is named herein a Twin Tip Ball Based Probe with retraction Loop or “Twin Tip BB Probe with Loop”.

Once the first portion of the tissue is treated (for example by using a blend mode of coagulation & cutting current), potentially aided by the loop being closed by a given force, the treated tissue may tear and move sideways, enabling new untreated tissue to come in contact with the electrodes. Such a process for treating tissue and closing the loop may potentially continue until the leg of the polyp is completely treated or cut and the polyp can be removed from the body safely.

In some case, if the physician closes the novel probe's loop using little force or apply no force to the loop during treatment, the tissue in contact with the electrodes may be coagulated and dehydrated in such a manner that tissue resistance to current flow rises. In such a scenario the generator may try to adjust current flow, but potentially no harm will be done to other body tissue, no such harm as described, by way of a non-limiting example, with reference to a short circuit presented in FIGS. 2F-2H.

The above-described process is potentially a safer process than a standard snare procedure done today with monopolar current flow. In such a monopolar scenario, and in case of high tissue resistance, the current may find a new path to flow from the snare's loop (active electrode) to the grounding pad (neutral electrode) located at the body's surface via any tissue with low resistance, for example the colon's wall. Such undesirable current flow might generate tissue damage and even colon wall perforation as rendered in FIGS. 2F-2H.

If the physician closes the novel probe's loop using excessive closing force during the treatment, the tissue in contact with the electrodes may coagulate and dehydrate in such a manner that the tissue mechanical strength reduces, the tissue become weaker, tears and moves sideways along the electrodes, potentially allowing untreated tissue to be treated. The above-described process is a safer process than a standard snare procedure done today with monopolar current flow probes. In such a monopolar scenario, when excessive force is applied to close a loop, some tissue may be treated in a partial manner, while other parts of the tissue may be treated properly, potentially resulting in post procedure bleeding as rendered in FIGS. 2I-2J.

A colonoscope may have a single working channel, or more than one working channel. A colonoscope typically includes at least one working channel with an inner diameter of 6.0 mm, 4.7 mm, 4.2 mm, 3.7 mm, 3.2 mm and 2.8 mm, and within a gastroscope an inner diameter of 2.8 mm, 2.6 mm, 2.2 mm, 2.0 mm and 1.2 mm, but are not limited to such exact dimensions. Some non-limiting example diameters of an outer diameter of a device as described here: 1 mm, 2 mm, 3 mm, 4 mm, 5 mm, 6 mm, and larger.

As rendered in FIG. 3A—The probe inserted in such a colonoscope working channel has an outer diameter 4D and the working channel has an inner diameter 14D. The inner diameter of the working channel is at least a little wider than the outer diameter of the probe. Such dimension differences yield a gap between the probe and the working channel. The gap is optionally defined as Gap=0.5×(14D−4D). Such a gap can typically be of 0.02 mm, 0.05 mm, 0.08 mm, 0.1 mm, 1.2 mm, 1.5 mm, 1.7 mm and 2.0 mm, but is not limited to such dimensions. Such gap dimensions are important to a design of the probe: (i) if the gap is too small e.g. 0.001 mm, friction between the probe and working channel might lock the probe and the working channel during insertion of the probe into the working channel, and; (ii) if the gap is too big e.g. 2.2 mm within a working channel with inner diameter of 6.0 mm, the probe outer diameter will be set to just 1.6 mm, yielding a too-small probe that may be harder than necessary to design and manufacture.

A laparoscopic port (trocar) may have a single working channel or more than one working channel. The laparoscopic port typically includes one lumen with an inner diameter of 15 mm, 12 mm, 10 mm, 5 mm and 3 mm, but is not limited to such dimensions.

Sizes of the spherical electrodes in the probe described above are limited due to the colonoscope inner working channel diameter. For example a 3.7 mm working channel may enfold a 3.2 mm probe. and such a probe's tip may house spherical electrodes with an outer diameter of 1.6 mm. Due to the size of the opening 26 and due to the spherical electrodes being electrically separate and in order to reduce potential of short circuit, a distance between the electrodes may be reduced to 1.0 mm and even to 0.8 mm, by way of a non-limiting example.

Such a probe's loop may be fabricated in such a manner that it does not conduct electrical current. The loop can be fabricated, by way of a non-limiting example, from a metal cable, and/or enfolded within a PTFE pipe (Teflon) or a full cross section polyurethane tube.

Potential Benefits of the “Bring to Me” Snare Concept

Many factors influence the medical results of polypectomy, among them; physician experience and technique, electric current properties, type of snare used, colon size and polyp tissue morphology.

One key for a successful polypectomy is optimal current delivery to treated tissue, where the loop design influences the treatment phase. A snare loop is often fabricated from a stainless steel cable. Cable properties, for example, may include cable diameter, cable material, type of twisting, number of wires within the cable and a type of coating. These properties may affect the efficacy & safety for the procedure during a current delivery stage. For example, a snare fabricated from a thin cable yields a higher current density than a thick cable that yields low current density (e.g. a cable with a cross section of 0.3 mm vs 0.6 mm). A thick cable may allow a faster & safer procedure than a thin cable, given same tools and generator setting. The snare cable may be with a diameter ranging from 0.3 mm to 1.0 mm OD, but not limited to such diameter.

Another key for a successful polypectomy is an optimal 3D size and shape of a snare or loop, influencing an ease of placing a loop on a polyp—the placing phase. The 3D size and shape of a loop is determined during the manufacturing stage of the loop. Such loop shape may be, for example, round, oval, hexagonal and asymmetrical. Such loops may be designed, for example, to be; flexible or rigid against side forces, stiff or flexible against forces in an axial direction. The 3D shape and the mechanical properties of the loop potentially affect a physician's ability to place the snare loop over a polyp head.

Snares used today, and the monopolar snares among them, are limited due to design constraint enfolded within the snare's loop. As described above, such constraints are divided in two main groups (i) current delivery aspects influencing the treatment phase (ii) 3D size and shape aspects influencing a placing of the loop on the polyp, the placing phase. To date snare design is a compromise between best current delivery properties and best shape enabling easy placement.

Some example embodiments of a probe presented herein enable a design without such compromise, since (i) the current delivery aspects are based on an electric design and do not affect the loop properties and; (ii) the loop design is based on an optimal 3D size and shape, enabling an optimal placement of the loop on the polyp and do not affect the current delivery properties.

For example, in some embodiments the grasping tool may be manufactured from a non-conductive material. In some embodiments, the cutting tool may be manufactured from a conductive material. In some embodiments the cutting tool may be manufactured from a flexible cable, which complies with tissue curves.

Probe with a Wider Working Diameter than Working Channel Diameter

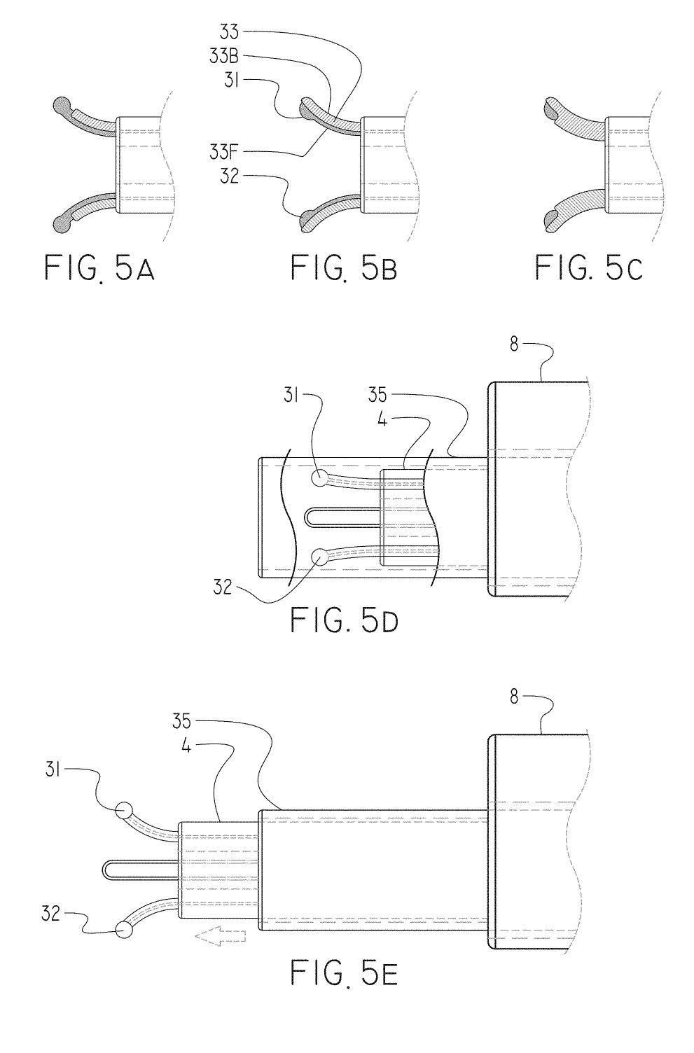

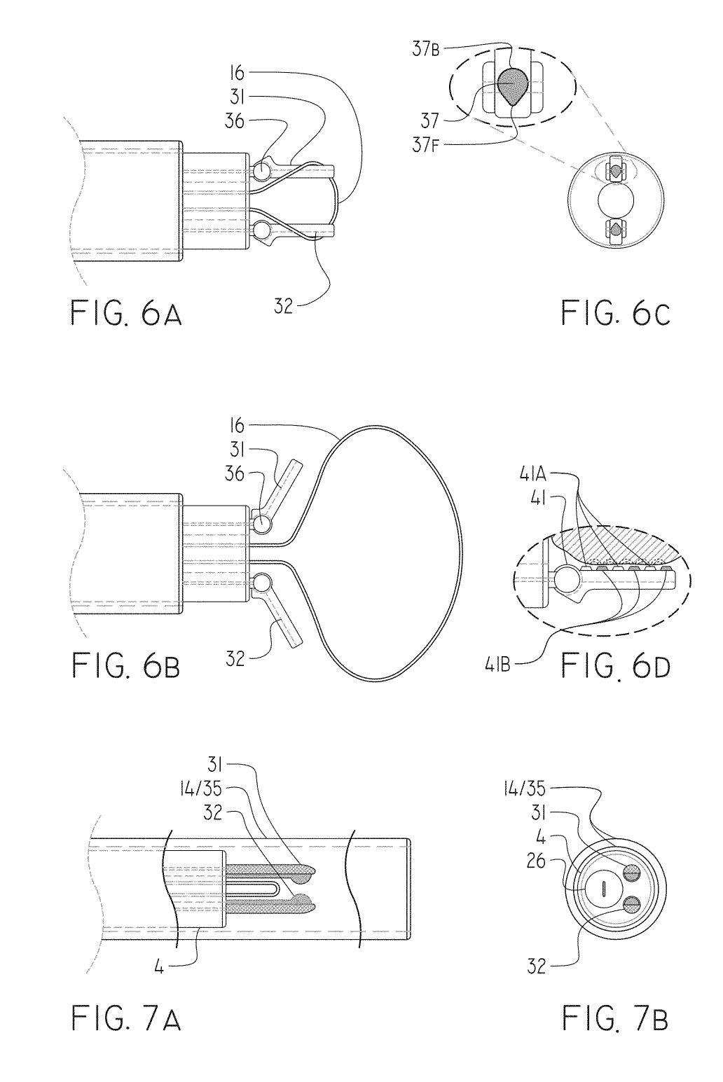

Above-mentioned U.S. Patent Application Publication number 2013/0006234; FIG. 5, describes an endoscope. Above-mentioned U.S. Pat. No. 5,630,813; FIG. 6 describes a balloon based expandable probe that may be inflated after such probe has been inserted in to the body's cavity. U.S. Pat. No. 6,228,083 describes a laparoscopic bipolar electrosurgical instrument.

Example embodiments of the probe described herein may use different electrode designs, enabling an insertion of a snare via a limited size working channel in to a colon. In some embodiments the tip of the Twin Tip BB Probe comprises the two electrodes, as can be seen in FIG. 2A. This yields a small treatment area. The example embodiment of a probe as presented in FIGS. 3A-3D functions well on tissue but the process may be slow. Due to a small functional distance between the two electrodes a “short circuit” may potentially occur, potentially reducing the efficacy of the process.

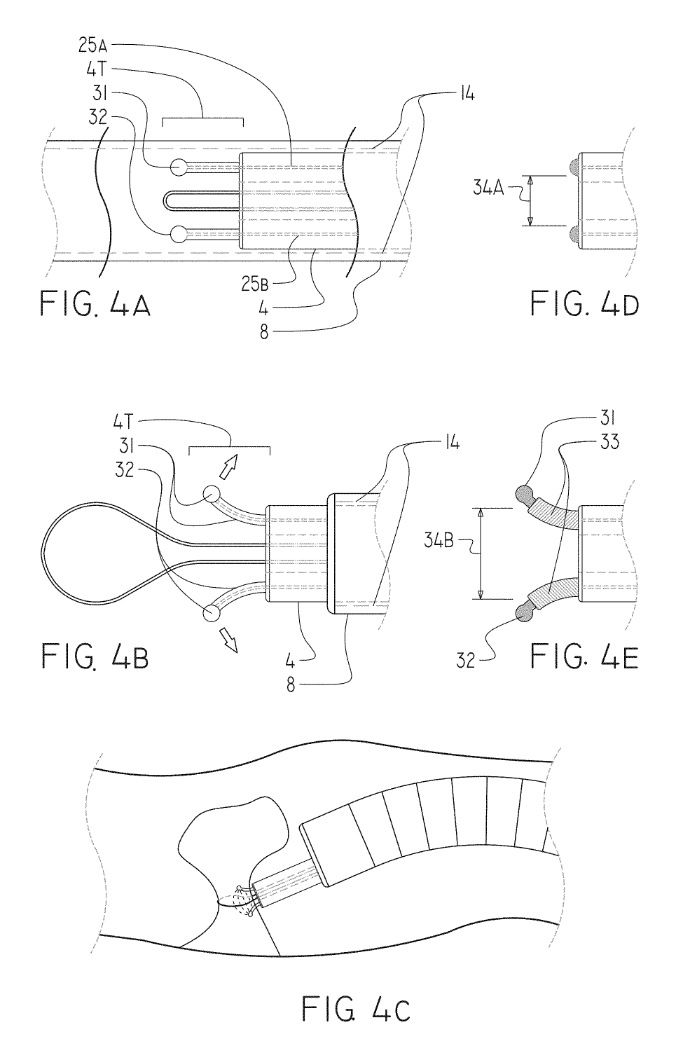

Reference is now made to FIGS. 4A-4C, which are simplified schematic illustrations of a probe according to some example embodiments of the present invention.

Reference is additionally made to FIGS. 4D-4E and FIGS. 5A-5C, which are simplified schematic illustrations of a probe according to some embodiments of the present invention.

As rendered in FIGS. 4A-4E, a probe 4 includes electrodes 31, 32 which optionally move sideways once a tip of the probe 4T protrudes from an opening of the colonoscope 8 working channel 14. The optional sideways movement potentially enables design of long electrodes which, once open sideways, can treat larger tissue areas with a larger functional distance 34B than a functional distance 34A generated by the Twin Tip BB Probe with Loop when both are introduced via a same inner diameter working channel. As rendered in FIG. 4E, one or both of the electrodes may include a shielding cover 33 enhancing the functional distance between the electrodes. In such a manner potential recurrence of a short circuit can be reduced dramatically. Such electrodes 31, 32 may be connected via conductive wires 25A, 25B to the generator (not shown).

FIG. 4A renders a locally exposed cross section view probe within the colonoscope and working.

As rendered in FIGS. 5A-5C, such electrodes 31, 32 may be shielded on one side with a non-conducting shield 33 (the shielded side being named a back of the electrode 33B) and not shielded on another side (the unshielded side being named the face of the electrode 33F). Such a design potentially enhances probe safety, keeping tissue which may be in contact with a back of the probe safe while performing a treatment of tissue by the front face 33F of the electrode.

FIGS. 5A-5C render three example embodiments for such designs, but the shielding 33 of the electrodes is not limited to such designs.

Reference is now additionally made to FIGS. 5D-5E, which are simplified schematic illustrations of a probe according to some example embodiments of the present invention.

Such electrodes may be self-expandable, moving aside based on their design, or expanded by a physician once a probe is introduced in to a body cavity. Design for such sideways expansion may include a spring based design, a mechanical mechanism, an electrical or thermal-based mechanism or a combination of more than one mechanism and/or method. The probe's electrodes may optionally be manufactured in an open manner, and prior to introduction of a probe tip to a colonoscope 8 working channel the electrodes may optionally be forced closed using an over tube 35, as rendered in FIGS. 5D-5E.

Once introduced to the colon the physician may optionally slide the over tube 35 backwards using probe handles, and the electrode are then free to self-expand sideways. Rendered in FIG. 5D, the probe's 4 two or more self-expandable electrodes 31, 32 are optionally housed within the over tube 35 in a closed manner, wherein FIG. 5E renders the electrodes in an expanded manner after the probe 4 has been optionally slid forward and the over tube 35 optionally kept in place.

FIG. 5D renders a locally exposed cross section view probe within the colonoscope and working channel.

Reference is now made to FIGS. 6A-6B, which are simplified schematic illustrations of a probe according to some example embodiments of the present invention.

As rendered in FIGS. 6A-6B—the probe's electrodes may be manufactured in an open manner. By way of a non-limiting example, the electrodes may be connected to the probe's shaft via a rotational hinge 36 and the electrodes may be spring loaded to open. Prior to introduction of the probe tip in to the working channel the loop 16 may be closed on the two electrodes 31, 32 acting as a temporary band. Once introduced to a colon a physician optionally slides the loop 16 forward, optionally using probe handles, thus opening the loop 16, and making the electrodes free to self-expand sideway 31, 32. The loop 16, which released the electrodes, may also be used for grabbing and pulling a polyp toward the open electrodes 31, 32. Retraction of the probe via the working channel or the over tube opening closes the probe's electrodes enabling extraction of the probe from the colon.

In another example embodiment of a probe, the electrodes may be fabricated from a material sensitive to temperature changes, or a bi-metal. When kept at room temperature, for example 21 C, the electrodes are in a closed formation, and when introduced to a body, for example at a temperature of 37 C, the electrodes may open sideways. Use of electrical current to treat the tissue may be used to pre-heat the electrodes, potentially generating the desired sideways movement.

Electrode 3D Design & Electrode Coating

Reference is now made to FIG. 6C, which is a simplified schematic illustration of a probe according to an example embodiment of the invention.

One task for a probe's electrodes is to enable current flow via the treated tissue. A second task of such electrodes may include cutting, grasping, pushing, moving parts of tissue or retrieving parts of the tissue to outside of a patient's body. For this second task the probe's electrodes may be designed, for example, as knife like electrodes, as grasper-like electrodes, as flat face electrodes, as round face electrodes, as electrodes with grasping grooves, as electrodes with one way teeth, and other designs.

An example embodiment of self-expandable electrodes may optionally enfold a knife shaped electrode 37, wherein a front face of the electrode 37F may be sharp and a back face of the electrode 37B may round is depicted in FIG. 6C.

Reference is now made to FIG. 6D, which is a simplified schematic illustration of a probe according to an example embodiment of the invention.

As rendered is FIG. 6D—the probe has been described so far with two electrodes, where each electrode connects via a cable to a generator (for example; one acts as a neutral electrode and another acts as an active electrode). Each of the two electrodes may be designed differently, for example each electrode 41 may be sub-divided as micro electrodes 41A, 41B. For example a first micro electrode may be an active micro electrode 41A, second micro electrode is may be a neutral micro electrode 41B, a third micro electrode may be an active micro electrode 41A, a fourth micro electrode may be a neutral micro electrode 41B, and so on. A potential benefit of such design is a precise control of current flow within the treated tissue.

In some embodiments an electrode coating potentially enhances probe performance. The electrodes may optionally be fully or partially coated. The electrodes may optionally be coated with more than one coating. The coating(s) may have various properties, such as, by way of some non-limiting examples: enabling low friction or high friction at the electrode surface, providing a non-stick coating, providing a coating for reducing or enhancing current flow between the electrode surface and tissue, providing a coating with tissue therapeutic properties, and other types of coating such as are known in the medical device industry.

Asymmetric Probe

A Twin Tip BB Probe with Loop is rendered in FIGS. 3A-3D. FIG. 3B depicts a front view of the probe tip, in which two spherical electrodes and a round opening enabling introduction of a loop are aligned along one vertical axis.

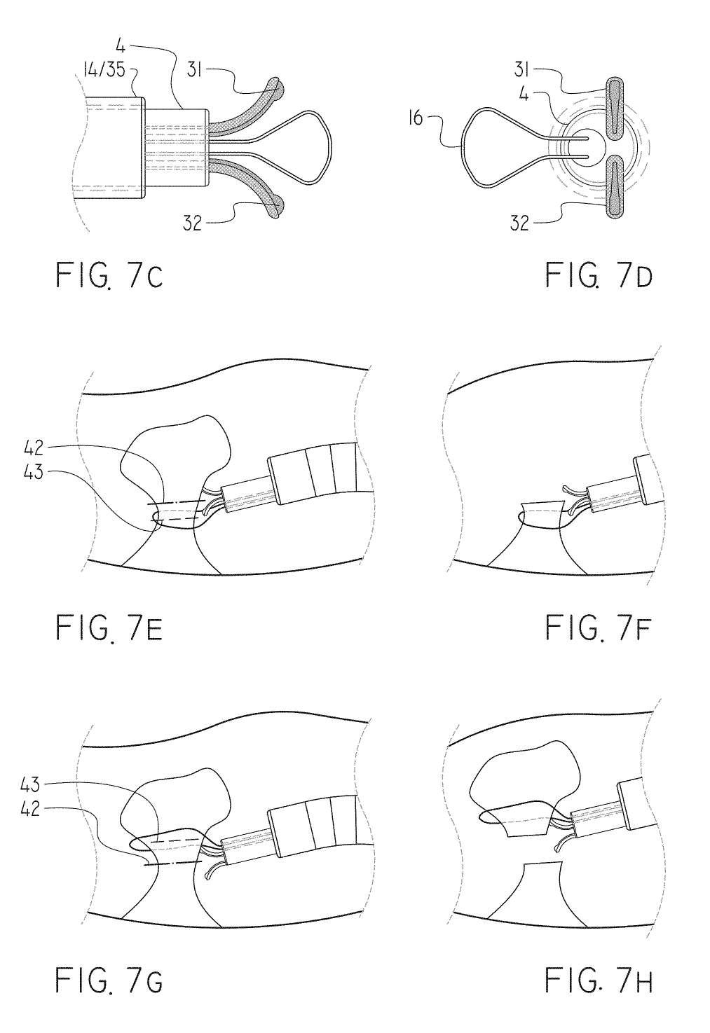

Reference is now made to FIGS. 7A-7H, which are simplified schematic illustrations of a probe according to some example embodiments of the invention.

As rendered in FIGS. 7A-7H—a novel probe 4 is inserted into a colon via the colonoscope working channel 14 and/or via an over tube 35. The probe tip enfolds two electrodes 31, 32 which can move sideways once protruding from a tip of the working channel 14 or a tip of the over tube 35. FIG. 7B depicts a front view of the probe tip in which the two electrodes 31, 32 and the opening 26 for introduction of a loop; are not aligned along one vertical axis. They are located in a manner to reduce an overall size of the probe tip.

The two electrodes 31, 32 form a tissue cutting line 42 which does not co-align with a plane of the loop 43. Such non co-alignment is potentially useful, potentially enabling a cutting of tissue while the loop grasps a leg of a polyp during the treatment, and optionally even after the treatment is done.

FIG. 7A renders a locally exposed cross section view probe within the colonoscope and the working channel or over-tube.

As Rendered in FIGS. 7E-7F, the cutting line 42 is locates above the loop in a manner which, after a polyp leg has been cut, the loop keeps holding the polyp leg stump, similarly to a tourniquet, potentially eliminating a recurrence of bleeding via the stump. Once the physician optionally decides to unleash the loop from the stump the physician may optionally loosen the loop using the probe's handle.

As Rendered in FIGS. 7G-7H the cutting line 42 is located below the loop in a manner which, after a polyp leg has been cut, the loop keeps holding the polyp head, potentially enabling easy retrieval of the polyp head out of the body. Polyp retrieval is highly desirable, enabling examination of a pathology of the tissue. It is common today, using a standard snare, that a polyp head falls in the colon and the physician has to use additional tools and time to retrieve the polyp head. Example embodiments of the probe described above potentially solves such a problem.

Steering the Loops

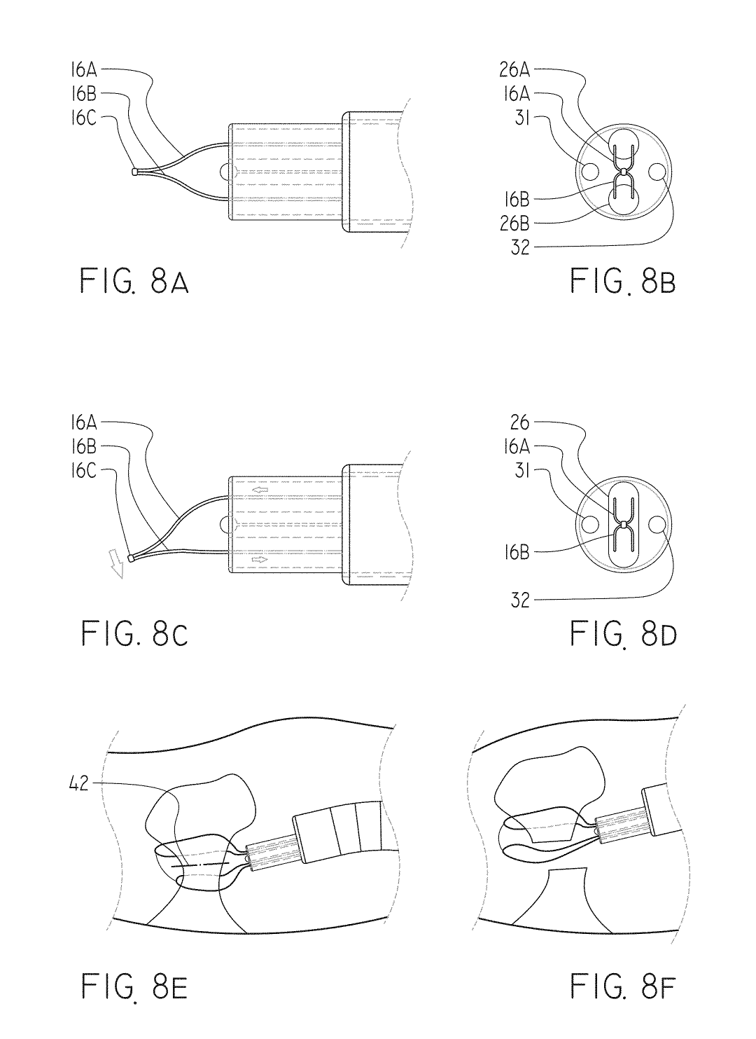

Reference is now made to FIGS. 8A-8F, which are simplified schematic illustrations of a probe according to some example embodiments of the invention.

As rendered in FIGS. 8A-8F—a probe is inserted into a colon via the colonoscope working channel. The probe tip enfolds two electrodes 31, 32 and two loops 16A, 16B. The loops 16A, 16B may be located in a single lumen 26, as presented in FIG. 8D, or in a double lumen 26A, 26B as presented in FIG. 8B. The two loops 16A, 16B may be connected at their distal side 16C, by way of a non-limiting example by a single point connection, by a linear connection, by a multipoint connection, or by a remote connection as presented in FIG. 8E. Each loop is connected to an external handle; for example, the upper loop connects to a handle termed an Upper Handle (UH) and the lower loop connects to a handle termed a Lower Handle (LH). The physician may optionally use both handles in the same manner, e.g. by pushing the UH and LH together, thus generating an opening of the loops symmetrically, as presented in FIG. 8A. The physician may optionally use both handles in a different manner, e.g. by pushing the UH and pulling the LH simultaneously, thus generating a downward movement of the loop tips as presented in FIG. 8C. Such downward movement may potentially assist the physician grabbing the polyp with the probe loops.

Such a probe enfolding two loops potentially produces additional benefits, for example; (i) during treatment of tissue, the loops may be closed both above and below the cutting line 42. Once cutting is complete the lower loop 16B may be loosened and the upper loop 16A may be kept closed. In such a manner the polyp is held at all times by the probe and can be retrieved easily. (ii) Such an array of loops and electrodes potentially enables easy feeding of the tissue into the electrode, treatment, area, ensuring fast & safe treatment.

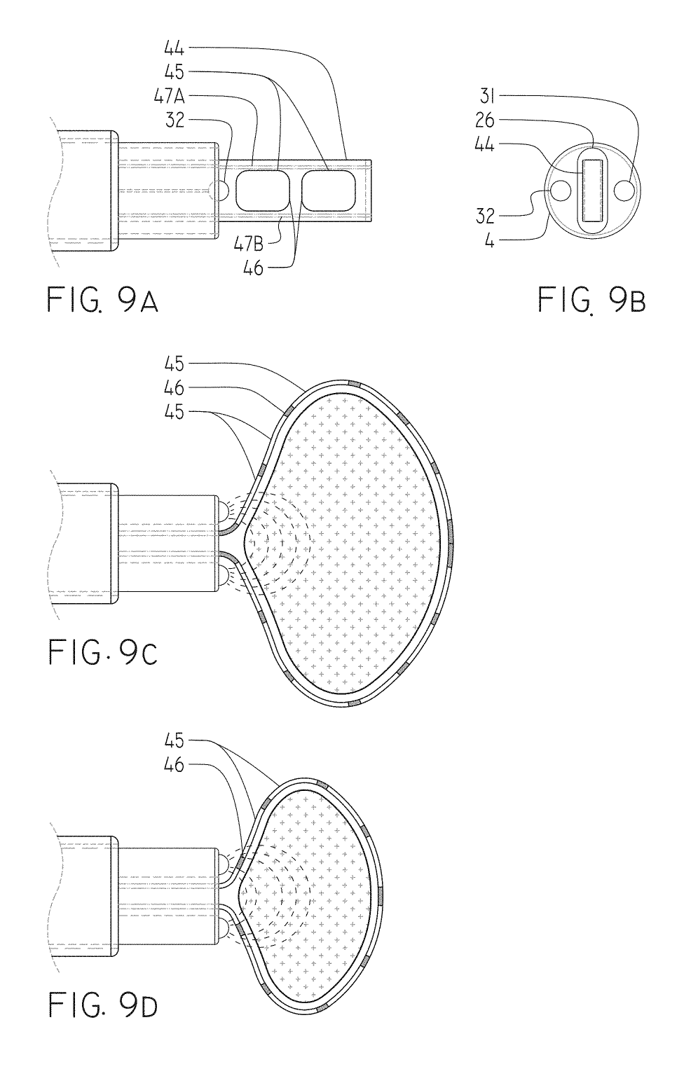

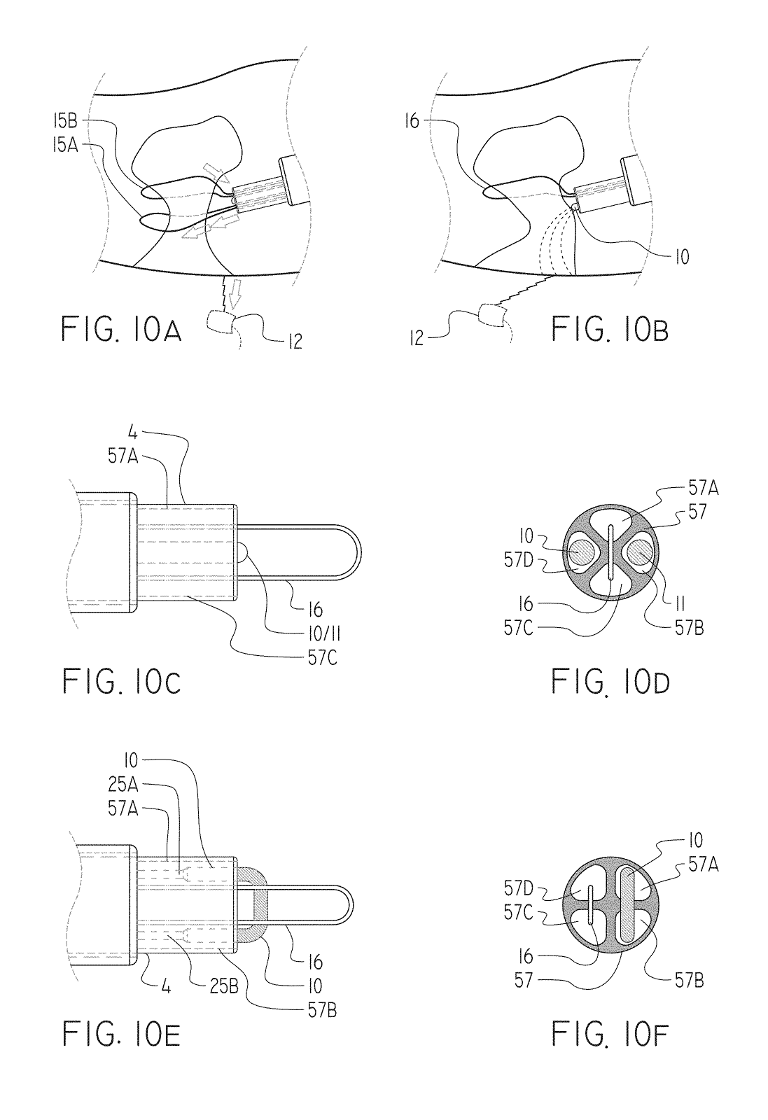

Reference is now made to FIGS. 9A-9D, which are simplified schematic illustrations of a probe according to some example embodiments of the invention.

As rendered in FIGS. 9A-9D—a probe 4 is inserted into a colon via a colonoscope working channel. The probe tip enfolds two electrodes 31, 32 and one lumen 26, enabling insertion of a loop 44 wherein such a loop 44 is optionally shaped as a curved surface, or as a ribbon. Such a ribbon may enfold one, two or more reinforcement wires referenced as 47A, 47B. As rendered in FIG. 9A the ribbon enfolds two reinforcement wires 47A, 47B. Such wires may be activated to open & close the ribbon or steer the ribbon in an upward or a downward direction similar to steering the probe described in FIGS. 8A-8F.

Such a ribbon may be with full coverage or with holes 45, the holes 45 generating partial coverage as rendered in FIGS. 9A, 9C, and 9D. When a physician activates a generator tissue treatment begins, and during such treatment the physician may close the ribbon. During such a closing process the ribbon may be moves closer to the electrodes and current flow may find various paths of flow via the ribbon holes 45 and treated tissue, as rendered in FIGS. 9C-9D. The ribbon left between the holes 46 may optionally be designed to have a cross section such that current can flow in-between with minimal disturbance.

General, Current Distributing, Multi Lumen & Steering

It is noted that the terms current and current flow may refer to a cutting current, to a coagulation current, to a blended current or to some other formulation of electric energy delivered to tissue. Such energy may be conducted via electrodes or via other power emitting apparatus; including but not limited to electrical\RF energy, vibration, laser and/or ultrasound based energy, other types of thermal energy, or other types of tissue cutting or coagulating technologies.

Irrigation may be used in conjunction with the probe, wherein irrigating fluids may optionally be irrigated by the physician from external sources via the introducer (or probe) onto a treated area. Such fluid may be irrigated for example: via: a cavity between the probe and the working channel, a cavity within the probe, a cavity between the probe and the loop, or other cavities within the colonoscope. Such irrigation fluids may optionally be used to: clean a treated area, change conductive properties of tissue around the probe, change conductive properties between a probe tip and tissue and change a temperature of the probe tip or the tissue. The following fluids may optionally be used: salt-less water, normal water, salted water, saline or other types of fluids. Such fluids may change the conductive properties. For example salt-less water may yield lower conductivity wherein salted water may yield higher conductivity between the tip of the probe and the treated tissue. It is noted that such irrigation may be used in conjunction with all embodiments described in the present specification.

In some embodiments the probe loop or loops as described above may optionally be designed differently to produce other forms of probes. Such design changes may include: changing the loop's conductive properties to semi-conductive or fully conductive, changing the loop's conductive properties so that at least part of the loop is conductive and part of the loop within the colon is non-conductive, connecting the conductive loop to one of the generator wires, connecting the conductive loop to one of the electrodes.

In some embodiments a current distributing apparatus which may be used, for example, to generate the following current flows in a body; (i) the generator may optionally drive current via a current distributing apparatus so that part of the current may flow via a first electrode and some of the current may flow via the conductive loop (ii) both parts of the current may optionally treat the tissue (iii) the current may return via the second electrode to the generator.

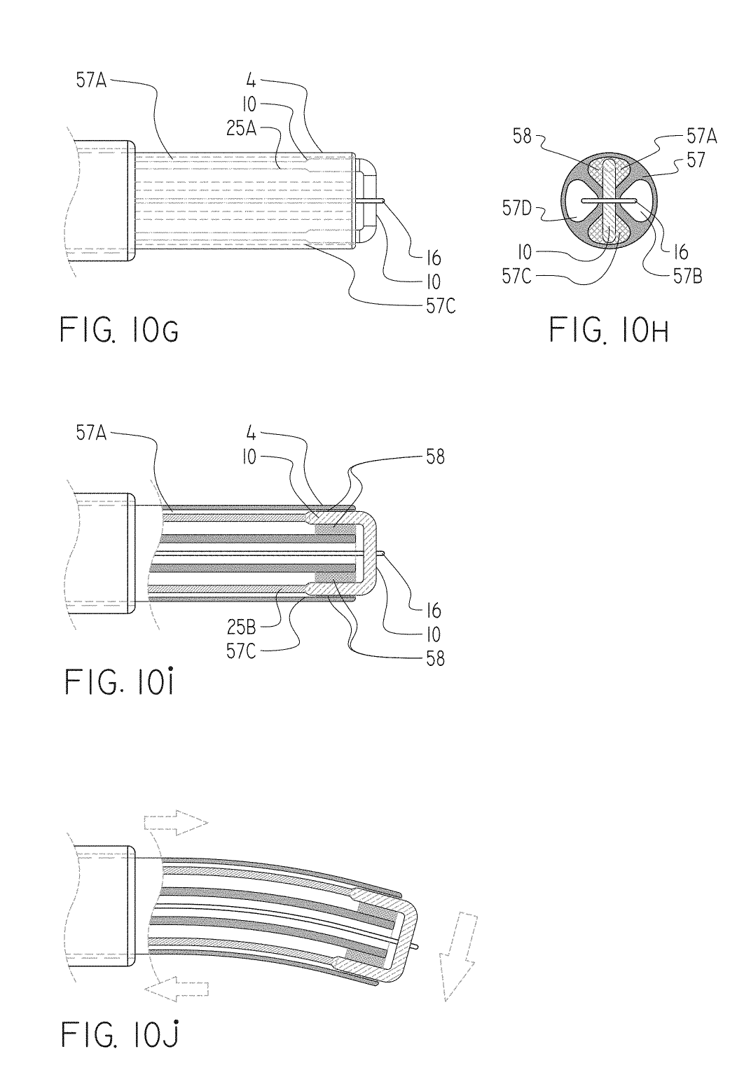

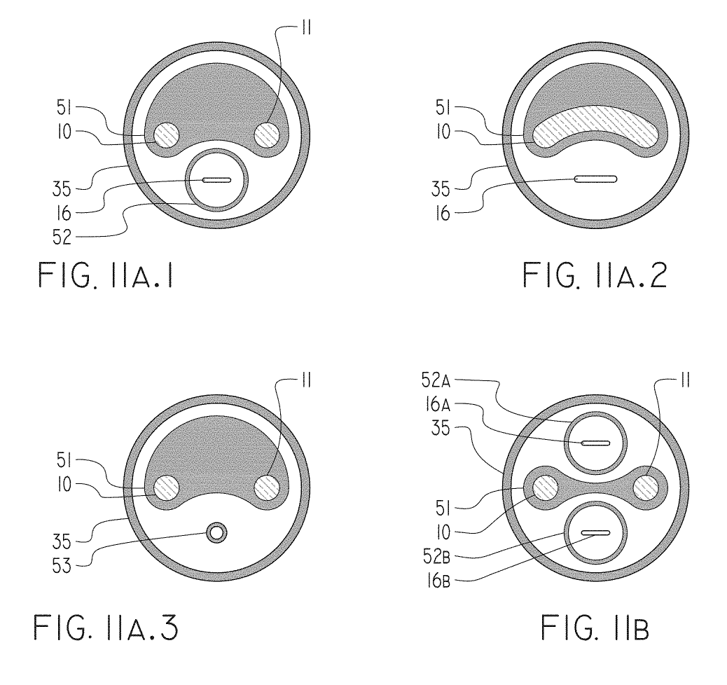

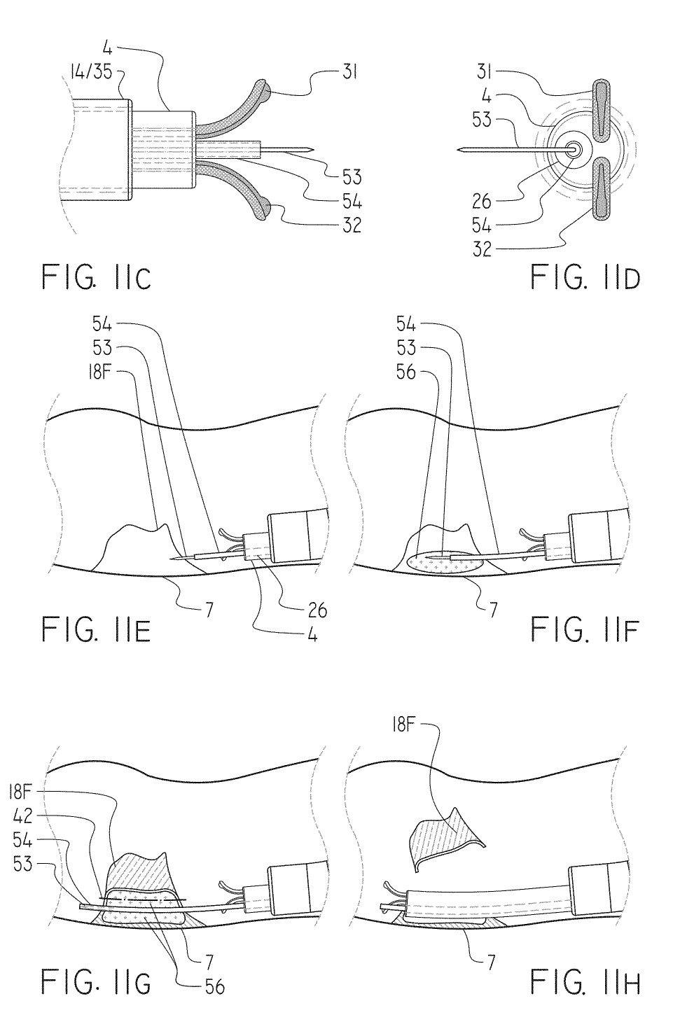

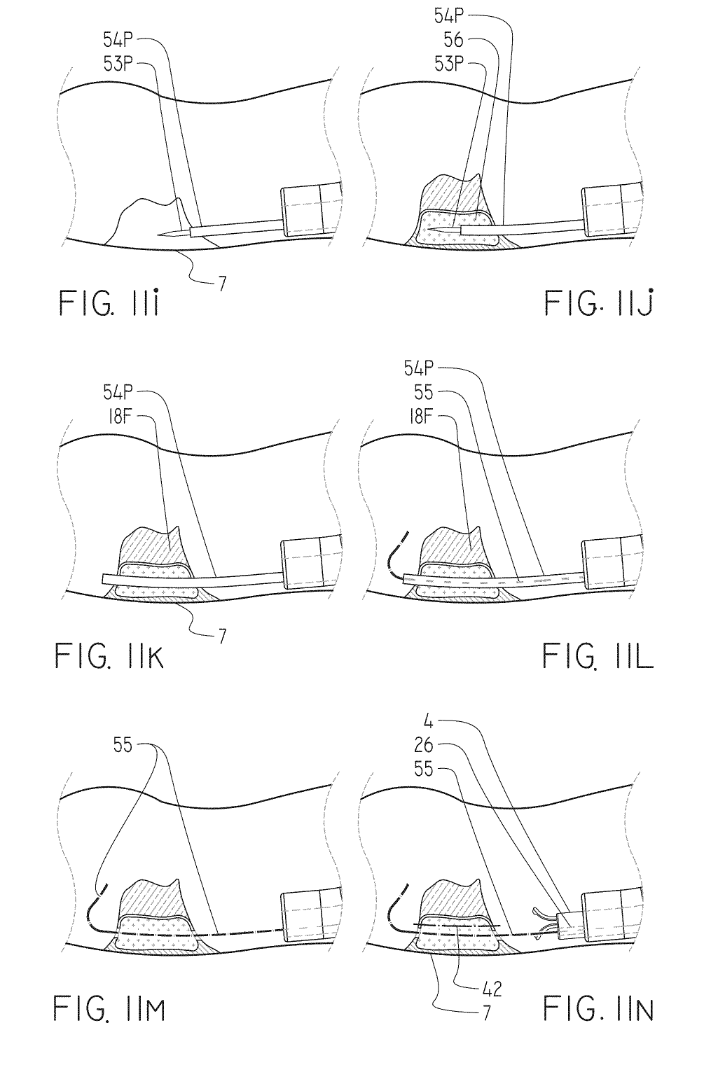

FIGS. 10A-10B are simplified schematic illustrations of a probe according to some example embodiments of the invention.

As rendered in FIG. 10A—another example is demonstrated; (i) the generator optionally drives current via the loop 15A and into treated tissue (ii) the current optionally treats the tissue (iii) part of the current optionally flows via a second loop 15B back to the current distributing apparatus and part of the current optionally flows via a grounding pad 12 to the distributing apparatus (iv) both portions of the current optionally flow back from the distributing apparatus to the generator (both not shown).

As rendered in FIG. 10B—a Twin Tip BB Probe with Loop may optionally be modified so that, for example: both balls acts as one active electrode 10, the loop 16 in made from a non-conductive material and the grounding pad 12 acts as a neutral electrode. In such a monopolar setting the loop 16 is optionally used to grab and move a polyp leg toward the active electrode, which is the two balls in this example, but is not limited to such an electrode configuration, and the active electrode may be used to apply current to the treated tissue. The current may then flow to the grounding pad and on to the generator (not shown).

Reference is now made to FIGS. 10C-10J, which are simplified schematic illustrations of a probe with a multi lumen pipe according to some example embodiments of the invention.

As rendered in FIGS. 10C-10D—a probe includes a multi lumen pipe 57. In the example depicted in FIGS. 10C-10D the multi lumen pipe optionally enfolds four sub lumens 57A-57D, although it is noted that the multi-lumen pipe is not necessarily limited to such a division. The probe 4 enfolds (i) two electrodes 10, 11 in the sub lumens 57B, 57D respectively and; (ii) a loop 16 that passes from the probe's handle (not shown) via the sub lumen 57A, the tip of the probe, the sub lumen 57C and back to the probe's handle. Such a probe enables usage of a small cross-section with a limited probe outer diameter such that the electrode tips are set across sides of the loop. Each wire, electrode and loop is potentially free to move linearly within the sub lumen, such movement optionally generated by a user via the probe's handle(s).

FIGS. 10E-10F depict a probe which includes a multi lumen pipe 57, where the multi lumen pipe enfolds four sub lumens 57A-57D, although not limited to such a division. The probe 4 enfolds (i) an electrode 10, that connects using two conductive wire 25A, 25B which pass in the sub lumens 57A, 57B respectively and; (ii) a loop 16 which passes from the probe's handle (not shown) via sub lumen 57C, the tip of the probe, the 57D sub lumen and back to the probe's handle. Such an example embodiment of a probe enables usage of a small cross-section with a limited probe outer diameter, wherein the electrode tip is set in parallel to the loop. Each wire, electrode and loop is potentially free to move linearly within the sub lumen, such movement optionally generated by a user via the probe's handle. Such a probe may be included with a monopolar electrode as rendered in FIGS. 10E-10F, or with a bipolar electrode.

FIGS. 10G-10J depict a probe which includes steering capabilities enfolded within the electrodes conductive wires. Such a probe comprising a multi lumen pipe 57, wherein in such example the multi lumen pipe enfolds four sub lumens 57A-57D but is not limited to such division. The probe 4 enfolds (i) an electrode 10 coupled to the tip of the probe, for example with glue 58 (ii) each tip of the electrode may be connected to a conductive wire 25A, 25B which passes from the probe in the sub lumens 57A, 57C respectively and; (iii) the conductive wires are potentially free to move linearly within the sub lumens and; (iv) a loop 16 which passes from the probe handle (not shown) via sub lumen 57B, the tip of the probe, the 57D sub lumen and back to the probe's handle. The loop is potentially free to move in a linear manner within the sub lumens. Such a probe optionally uses the conductive wire 25A, 25B in two manners (a) passing electrical current to treat a tissue and; (b) steering the probe tip by pulling back one conductive wire and pushing another. Such pushing\pulling generates a steering of the tip of the probe, enabling the physician to perform a maneuvering of the device tip during the procedure. Such pushing\pulling action may be generated by the external handle (not shown).

The multi lumen described above may be manufactured from flexible or rigid materials, current conductive or non-current conductive materials.

Electrode Housing, Guide-Wire & Needle

Reference is now made to FIGS. 11A1-3 and 11B, which are simplified schematic illustrations of a probe according to some example embodiments of the invention.

FIG. 11A.1 depicts a probe which includes an over tube 35 which enfolds (i) two electrodes 10, 11 enfolded in an electrode housing 51, where the electrodes are connected by wires to the generator (not shown) and; (ii) a small over tube 52 which enfolds a loop 16. Such an apparatus enables movement of its components in a linear and rotational manner, for example the small over tube 52 can be moved in a linear manner on the loop 16, potentially causing the loop to close or open. The loop 16 and the small over 52 tube can optionally be rotated within the over tube 35. The electrode housing 51 and its electrodes and wires can optionally be moved in a linear manner within the over tube 35. Such a design provides additional degrees of freedom to the physician, beyond simple forward and back, during a procedure.

FIG. 11A.2 depicts a probe comprising an over tube 35 which enfolds (i) one electrode 10 enfolded in an electrode housing 51 and; (ii) a loop 16. Such a design enables usage of a monopolar setting. Current optionally flows from a generator, via the electrode, the treated tissue, the tissue of the body, the grounding pad and back to the generator (not shown). The loop 16 potentially ensures that the tissue of a polyp leg keeps in contact with the electrode.

FIG. 11A.3 depicts a probe which includes an over tube 35 which enfolds (i) two electrodes 10, 11 enfolded in an electrode housing 51 and; (ii) a hollow needle 53 or a guide-wire (not shown).

FIG. 11B depicts a probe including an over tube 35 which enfolds (i) two electrodes 10, 11 enfolded in an electrode housing 51, where the electrodes are connected by wires to the generator (not shown) and; (ii) a small over tube 52A enfolds a loop 16A (iii) a small over tube 52B enfolds a loop 16B. Such a probe enables movement of its components in a linear and rotational manner. A “figure eight” like design of the electrode housing 51 keeps each of the small over tube in place, and ensures easy linear and rotational movement of each of the small over tubes.

Probe using an “Over the Guide Polyp Removal” Maneuver

FIGS. 11C-11N are simplified schematic illustrations of a probe according to some example embodiments of the invention;

FIGS. 11C-11D depict a probe 4 inserted in a colonoscope working channel 14 and in a colon, including: an over tube 35 which enfolds (i) two electrodes which may optionally be self-expanding electrodes 31, 32 where the electrodes are connected by wires to the generator (not shown) (ii) a lumen 26 within the probe 4 which spans from the probe's tip to the external handle, where in such a lumen a guide wire (not shown), needle or needle 53 housed within a pipe 54 can optionally be introduced by a physician to the colon, optionally when the colonoscope has reached tissue which should be treated.

Such a probe can include other forms of electrodes (e.g. non self-expanding electrodes, spherical electrodes and other electrodes) which may be used in a bipolar or monopolar setting. Such a probe may even have only one electrode used in a monopolar setting. Such a probe can optionally be used with or without the over tube 35. Such a probe can be designed in a manner similar to FIGS. 11a1, where the loop 16A can be replaced with a needle. Such probe can be built in a manner similar to FIG. 11a1, wherein the loop 16A and small over tube 52A can optionally be replaced with a needle. Such a probe can be built in a manner similar to other embodiments presented herein, where the needle or guide-wire can optionally replace the loop in the probe; or the needle housed within the pipe can optionally replace the loop; or the needle or the guide-wire can optionally laid along such loop; or the needle housed within the pipe can optionally be laid along such loop. The needle as described in this section can optionally be replaced or laid along a guide-wire.

A physician may encounter several kinds of polyps within the colon. A pedunculated polyp comprises a tissue mass named a polyp head, which is connected by a thin tissue to the colon wall, the connecting tissue being named the polyp leg. Such a pedunculated polyp may be handled by the physician by steering the snare's loop over the head of the polyp and grabbing the polyp leg. Once the loop is firmly closed on the poly's leg the physician can activate the generator and remove the polyp.

A flat polyp (named also a flat adenoma or a sessile polyp) differs from the pedunculated polyp in its 3D structure and possibly other tissue features. The flat polyp looks like a small bump which rises from the colon wall. Such a flat polyp is potentially hard to grab with a snare loop due to its round edges and lack of height.

FIGS. 11E-11H depict a probe 4 inserted via the colonoscope working channel 14 i to the colon. Once the probe protrudes from the working channel or the over tube 35, the physician may introduce, via the probe's lumen 26, a needle 53 housed safely within a pipe 54.

(i) The physician may now have a good view of the flat polyp and the tip of the pipe 54 enfolding the needle 53, and may push the needle forward thus the needle will protrude from the pipe. Once the needle protrudes the physician may push the tip of the needle in to the flat polyp 18F and inject (e.g. fluids, saline, gas or water) in to the tissue. Such fluids may elevate the flat polyp from the colon wall, potentially generating a safety buffer between the colon wall and the treated tissue as rendered in FIG. 11F. The physician may optionally push the needle further, optionally until the needle punches out from the polyp tissue and protrudes beyond into the colon lumen. Once the needle and the pipe are located in the colon lumen, the needle may optionally be retracted in to the pipe for safety, as depicted in FIG. 11G.

(ii) The physician optionally uses the needle and pipe as a guide-wire and optionally slides the probe 4 on the pipe 55 during activation of the generator. Such a sliding along the guide wire can potentially generate a cutting line 42 which removes the polyp nicely. In such a manner the probe is potentially anchored against upward or downward movements during tissue treatment. Once the probe has completed treatment, as rendered in FIG. 11H, the physician may optionally retract the probe and retrieve the polyp. The same probe can optionally be used without the pipe 55, in which case the needle is enfolded within the probe's lumen 26.

A potential benefit of such an anchoring against upward or downward movements is now described. If a physician does not have an anchoring against upward movements he might not gain good contact between the electrode and the treated tissue, or might lose contact during treatment. If the physician does not have anchoring against downward movements the physician might move the electrode too deep into the colon wall, potentially generating colon wall trauma and even colon perforation.

It is noted that the probe and technique described above may also be used without fluid injection.

FIGS. 11i-11N depict a priming tool which optionally includes a needle 53P enfolded within a pipe 54P, which is optionally inserted via the colonoscope working channel in to the colon.

(i) When the priming tool protrudes from the working channel or the over tube, the physician may optionally introduce a needle into a flat polyp tissue and inject fluids 56 as described before. FIG. 11i depicts the flat polyp after the fluid injection. The physician may optionally push the needle still further, optionally until the needle punches out from the polyp tissue and protrudes into the colon lumen. When the needle and the pipe are located in the colon lumen, the needle may optionally be retracted in to the pipe for safety, as rendered in FIG. 11K.

(ii) The physician may optionally retract and remove the needle completely out of the pipe while keeping the pipe in the polyp tissue. The physician can hold a pipe opening which is located outside a body, which such pipe protrudes from the working channel opening close to the colonoscope handle. The physician can optionally insert a guide-wire 55 into the pipe 54 and push the guide-wire until the guide-wire protrudes from the tip of the pipe located in the colon, as rendered in FIG. 11L. When the guide wire introduction is done the physician may optionally remove the pipe 54 by sliding the pipe 54 back from the colonoscope working channel and the guide-wire, keeping the guide-wire in the polyp, as rendered in FIG. 11M.

(iii) The physician may optionally use the guide-wire and slide the guide-wire end, located outside the body, in to the probe's lumen 26. The probe 4 may slide on the guide wire 55 until the probes protrude from the working channel and in to the colon. Once the probe electrodes touch the tissue to be treated the physician can optionally activate the generator and slide the probe slowly on the guide-wire until the polyp have been removed. In such a manner the probe is potentially anchored against upward or downward movements by the guide-wire during tissue treatment. Once the probe has completed the treatment the physician may optionally retract the probe and retrieve the polyp.

A potential benefit of using such a priming tool is usage of a smaller probe lumen 26, since the only part sliding through the lumen is the guide-wire and not the pipe 54 and needle 53 as rendered in FIGS. 11E-11H.

It is noted that a similar probe and technique may be used without the fluid injection.

It is noted that the guide-wire can optionally be with non-conductive or conductive properties; and that the guide-wire may optionally be used as an electrode.

It is noted that the needle described herein may optionally be designed as: (i) a fully rigid needle; (ii) a first segment of a rigid needle connected to a flexible pipe, potentially enabling good tissue punching properties while keeping the needle's tail flexible or; (iii) a fully flexible needle. More over the needle may optionally be designed with various 3D shapes for example: (i) a straight needle (ii) a first segment of a curved needle connected to a straight needle, potentially enabling good tissue punching properties and also directing the movement of such a needle in tissue. By way of a non-limiting example an upward curved needle may generate upward movement of the needle within the tissue thus directing the needle far from the colon wall.

Probe used for “Over the Polyp Head” Maneuver

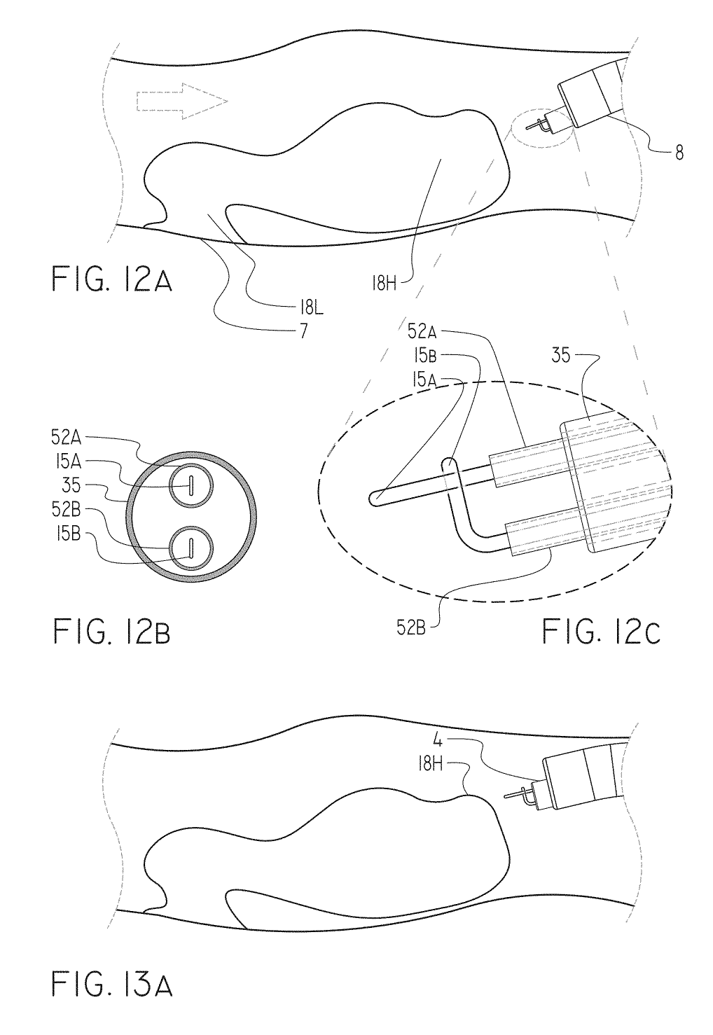

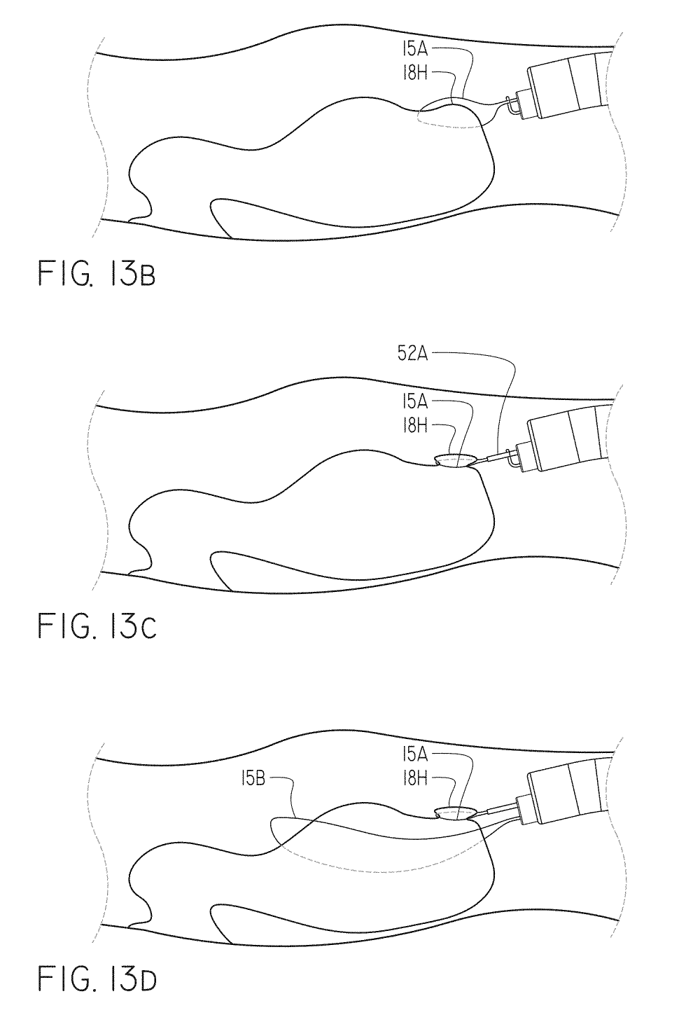

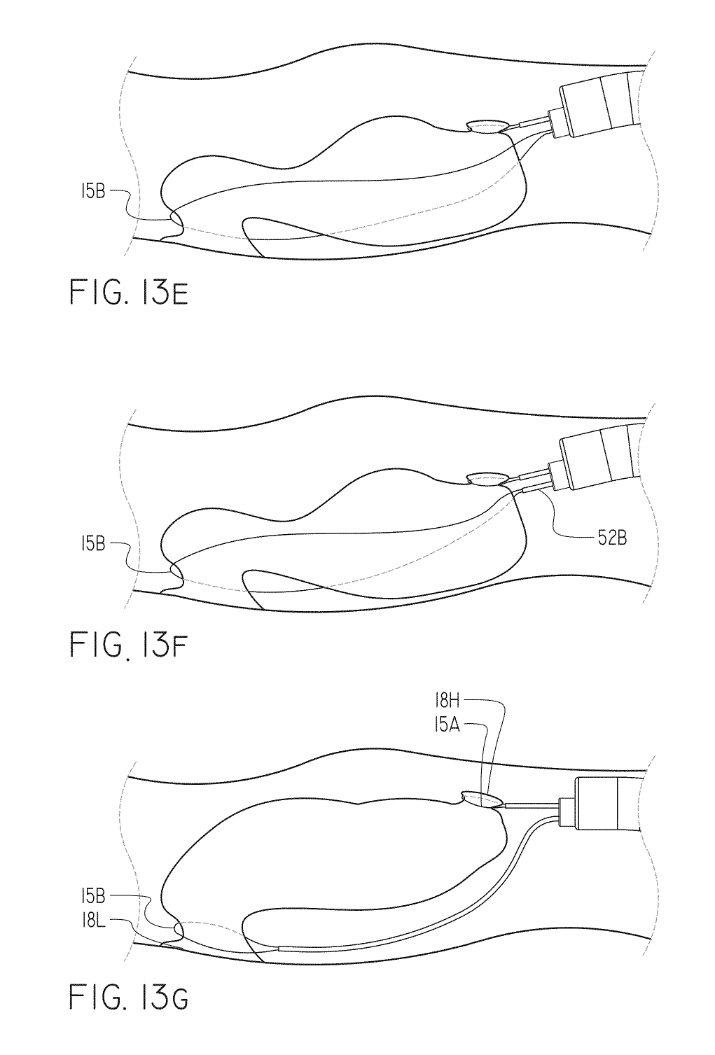

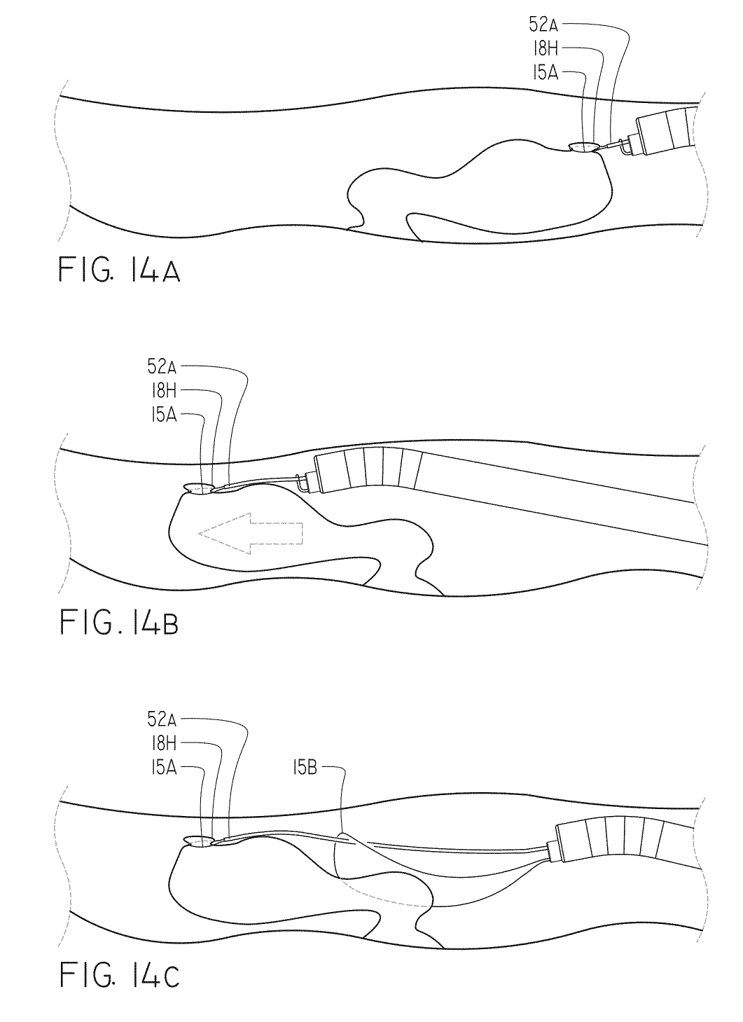

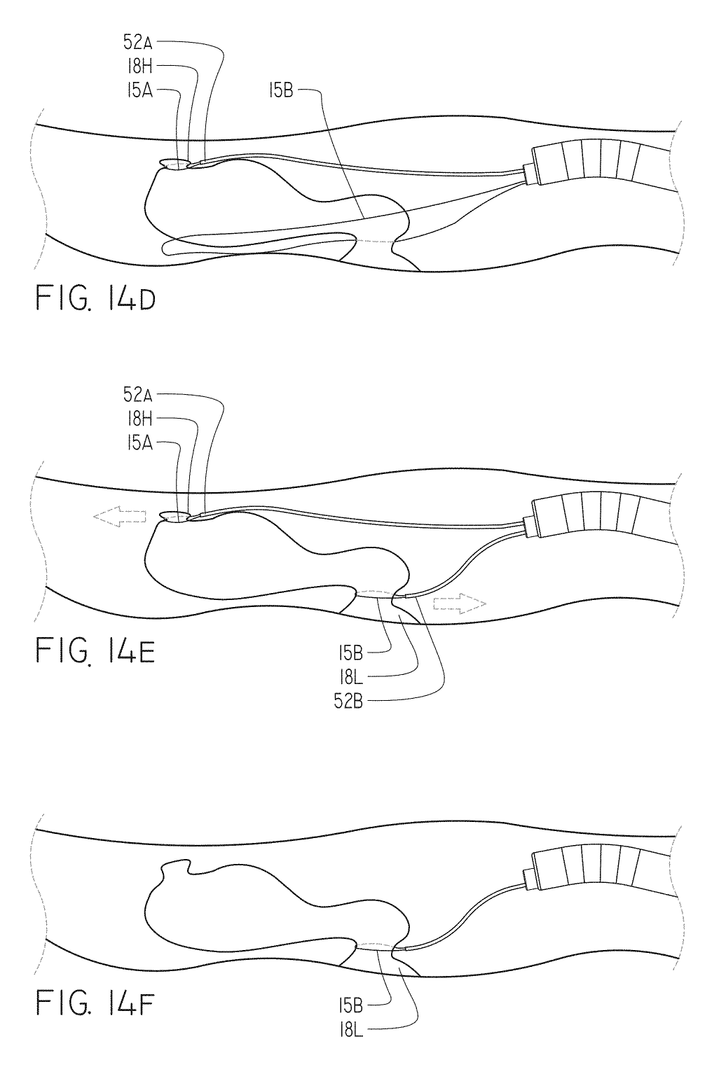

Reference is now made to FIGS. 12A-12C and 13A-13G, which are simplified schematic illustrations of a probe according to some example embodiments of the invention.

As rendered in FIG. 12A—some polyps grow to a significant size within the colon. Such polyps may range between 2 cm and 6 cm, but are not limited to such a size. Due to a limited diameter of the colon, the polyp may start spreading and lie sideways within the colon. For example due to movement of fecal matter from the cecum to the rectum, such a big polyp typically spreads downstream relative to the colon, as rendered in FIG. 12A. In some embodiments a colonoscope 8 travels upstream relative to the colon, inserted from the rectum and pushed, optionally until it reaches the cecum. When such a colonoscope 8 encounters a big polyp, the polyp leg 18L may be mostly hidden by the polyp head 18H from a camera (not shown) in the tip of the colonoscope.

In such a big polyp scenario the physician may sometimes be unable to observe the polyp leg 18L and might (i) cut the polyp to small pieces and remove each one of the pieces; (ii) push and steer the polyp around until the physician finds the polyp leg 18L; (iii) try to push a loop or snare in and grab the polyp leg 18L blindly; and (iv) send the patient to an open surgery.

As rendered in FIGS. 12B-12C—a probe 8 comprising an over tube 35 enfolds (i) a loop 15A enfolded in a first over-tube 52A; (ii) a loop 15B enfolded in a second over-tube 52B; and (iii) the first loop 15A passes through the second loop 15B. The loops 15A 15B may be used as electrodes in a bipolar manner. One loop might be used in a monopolar manner with a grounding pad. Two electrodes may be inserted as rendered in FIG. 11B. Prior to insertion in to the colon, the loops 15A 15B may be loaded as rendered in FIG. 12C, where the upper loop 15A passes through the lower loop 15B.

As rendered in FIG. 13A-13G—the probe as described above will be used to treat a big polyp within the colon in a method described as “over the polyp head”. The probe's loops as described in FIG. 12C may be loaded as described and;

(i) When the physician spots a big polyp as rendered in FIG. 13A the physician optionally loosens the first loop 15A enabling the first loop 15A to open. The physician optionally steers a tip of the colonoscope until the loop 15A has good contact with a surface of the head of the polyp 18H, as rendered in FIG. 13B. When contact is made the physician optionally closes the loop 15A, optionally by pushing the over tube 52A forward, or by pulling on an end of the loop 15A to close the loop 15A. The upper loop 15A potentially has a firm grasp of the polyp head 18H, or of any other part of the polyp, as rendered in FIG. 13C.

(ii) The physician optionally loosens the second loop 15B, enabling the second loop 15B to open, optionally forcing the second loop 15B to move over the first loop 15A and upstream into the colon, as rendered in FIG. 13D. When the second loop 15B has been opened, as rendered in FIG. 13E the physician optionally steers the colonoscope upwards, and optionally closes the second loop 15B by pushing the over tube 52B forward or by pulling on the second loop 52B.

(iii) As rendered in FIG. 13G the probe's loop 15B is optionally positioned in a proper manner on the big polyp leg 18L and treatment can be commenced in a monopolar or bipolar setting. Once the polyp leg is cut the physician can optionally retrieve the polyp using the first loop 15A which still grasps the polyp head.

In some embodiments the loop 15B forms a tissue cutting line which is not on a same plane as the loop 15A. Such non co-alignment is potentially useful, potentially enabling a cutting of tissue while the loop grasps a polyp during the cutting, and optionally even after the cutting is done.

In some embodiments coating potentially enhances grasping and/or cutting tool performance. The grasping and/or cutting tool may optionally be fully or partially coated. The grasping and/or cutting tool may optionally be coated with more than one coating. The coating(s) may have various properties, such as, by way of some non-limiting examples: enabling low friction or high friction at the electrode surface, providing a non-stick coating, providing a coating for reducing or enhancing current flow between the electrode surface and tissue, providing a coating with tissue therapeutic properties, and other types of coating such as are known in the medical device industry.

In some of the embodiments described herein with reference to various grasping and cutting tools shaped as a loop, the cutting tool may be shaped as a 2D or a 3D loop. Such a loop may be shaped, for example as, round, oval, hexagonal or even asymmetrical. Such a loop may be designed, for example, to be: flexible, or rigid against side forces, and/or stiff or flexible against forces in an axial direction.

In some of the embodiments described herein with reference to various grasping and cutting tools shaped as a loop, the loop may be manufactured of a stainless steel cable. The cable may be 0.2 mm to 0.6 mm in diameter, although larger and smaller diameters, such as 0.1 mm to 2 mm are contemplated. In some embodiments the cable may be formed as a single strand cable, or as a multi-strand cable in a configuration of 7×1, 7×3, 3×7, 7×7, 3×3, 19×1 or other type of cable configuration. In some embodiments the loop cable may be coated in part or along an entire length with a coating, for example affecting insulation, friction, shape, cross section and more.

In some of the embodiments described herein with reference to various grasping and cutting tools formed of wire, such tools may optionally be formed of conductive or non-conductive materials.

Probe used for “Grab and Push” Maneuver

Reference is now made to FIGS. 14A-14F, which are simplified schematic illustrations of a probe according to some embodiments of the invention.

As rendered in FIGS. 14A-14F a probe as described above is used to treat a bid polyp within the colon in a method described as “grab and push”. The probe loops, optionally as described with reference to FIG. 12C, are optionally loaded as described and:

(i) A physician spots a big polyp, and optionally loosens a first loop 15A enabling the first loop 15A to open. The physician optionally steer a tip of the colonoscope until the first loop 15A has a good contact with any surface of the head of the polyp 18H. Once the contact is good the physician optionally closes the loop 15A by pushing an over tube 52A forward. The upper loop 15A potentially grasps and has a firm hold of the polyp head 18A, or any part of the polyp head, as rendered in FIG. 14A.

(ii) The physician optionally pushes the colonoscope upstream with the probe's first loop 15A holding the head of the polyp 18H, optionally using the colonoscope vision to see that he moves it in a safe path and manner. Once the physician pushes the big polyp head all the way upstream the physician stops pushing, as rendered in FIG. 14B.

(iii) The physician optionally moves the colonoscope downstream while pushing the over tube 52A forward until a clear view of the polyp leg is seen. The physician optionally loosens and pushes a second loop 15B as rendered in FIG. 14C until the second loop 15B reaches above the polyp head, as rendered in FIG. 14D.

(iv) The physician optionally closes the second loop 15B, optionally by pushing the small over tube 52B forward until the second loop 15B is closed, or by pulling on the second loop 15B. If additional steering of the polyp is needed the physician may optionally push the first loop 15A and the over tube 52A forward, optionally until good visibility of the polyp leg is gained.

In some embodiments the physician may optionally treat, or cut, the polyp, as rendered in FIG. 14E, and in some embodiments the physician may optionally release the first loop 15A and use the second loop 15B during such treatment, as rendered in FIG. 14F.

If the physician fails to perform the task in a first attempt; the physician may release the tissue, retract the loops in to the over tubes 52A 52B and retract the probe out of the working channel, keeping the colonoscope in the colon. Once the probe is outside the body, the physician may optionally reload the loops as shown in FIG. 12C, insert the probe in to the working channel and try again. The probe may also optionally be reloaded within the body using loop controls enabling the physician to steer (e.g. move the loops in a liner manner—push or pull, rotational manner, and to close and open the loops) the loops 15A 15B one over the other.

A probe and methods as described above and elsewhere herein enable removal of a large polyp while keeping the colonoscope vision effective at all times, enabling the physician safe and effective big polyp removal.

As rendered in FIGS. 12-14 the probe enfolds two loops. Such loops may have current conductive or non-conductive properties. One of the loops may be with current conductive properties and the second loop may be with non-conductive properties.

“Multi-Lumen Probe used for Over the Polyp Head Maneuver”

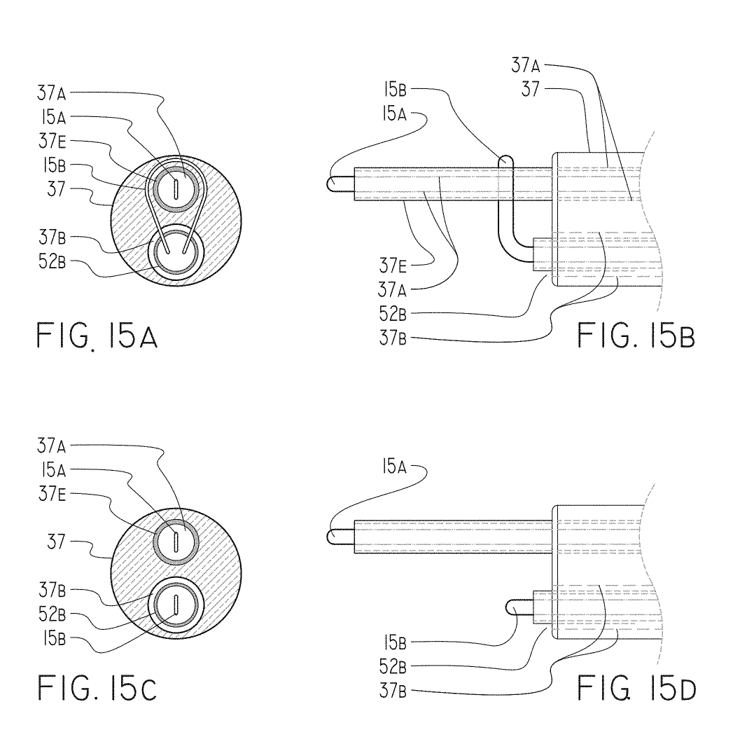

Reference is now made to FIGS. 15A-15D, 16A-16D and 17A-17F, which are simplified schematic illustrations of a probe according to some embodiments of the invention.

As rendered in FIGS. 15A-15D—a probe comprising a multi-lumen 37 which: (i) enfolds a first loop 15A enfolded in a first lumen 37A. Such a lumen and loop may pass also via a main multi-lumen 37 and a multi-lumen's extension 37E and; (ii) enfolds a second loop 15B enfolded in an over-tube 52B, wherein such an over-tube passes via the multi-lumen's second lumen 37B (iii) the first loop 15A passes through the second loop 15B, and the second loop 15B may also pass over the multi-lumen's extension 37E. In some embodiments the two loops 15A 15B may be used as electrodes in a bipolar manner or one loop might be used in a monopolar manner with a grounding pad. The loops 15A 15B are optionally preloaded as rendered in FIGS. 15A-15B, where the second loop 15B passes over the first loop 15A and over the multi-lumen's extension 37E. Such preloading may be done prior to insertion of the probe to a colonoscope working channel.

FIGS. 15A-15B show the probe in the loaded configuration, where the loops 15A 15B are passed one over the other and the probe is ready to be used. FIGS. 15C-15D show the probe in an un-loaded or post-procedure configuration where the loops 15A 15B are not passed one over the other, for example after a polyp has been cut and removed from a body cavity.

A direction termed herein as upwards is usually defined in colonoscopy as a relative direction relative to a location of a camera of a colonoscope is located at 12 o'clock, downward is usually defined in colonoscopy as the relative direction wherein the colonoscope working channel is often typically located, at 5 or 6 or 7 o'clock. Left and right are also defined relatively to the camera.

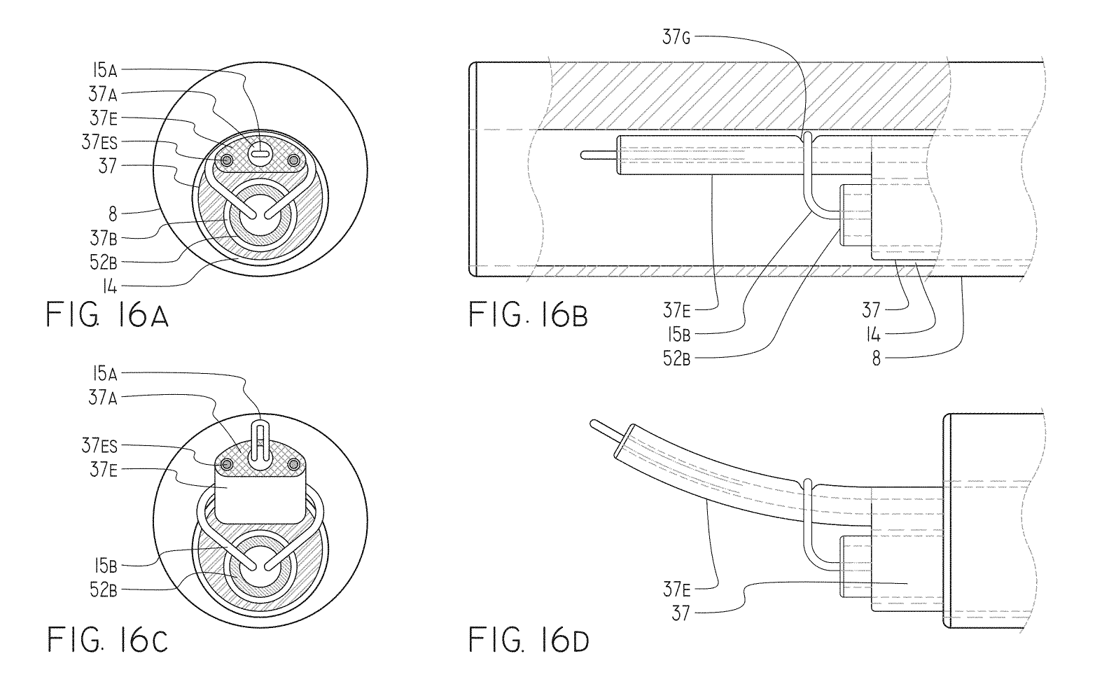

As rendered in FIGS. 16C-16D, a multi-lumen's extension 37E may be flexible and may be designed to bend upwards relative to a main axis of the probe. Moreover, the multi-lumen extension 37E may be designed to bend sideway relative to a main axis of the probe and/or the colonoscope. Such a bending property of the multi-lumen's extension may be due to (i) a nature of the multi-lumen's extension material; (ii) a way that such a multi-lumen extension is produced, molded or pre-treated; (iii) due to an optional pre-loaded spring 37ES placed within a multi-lumen extension and/or within the main multi-lumen 37; (iv) an addition of a pre-loaded spring placed and coupled on-top of (or under) the multi-lumen extension and/or the main multi-lumen (v) other devices which are optionally used to bend the multi-lumen extension by an operator, such as, by way of a non-limiting example, by pulling a cable which passes through the multi-lumen extension, and the multi-lumen up to an external handle.

As rendered in FIGS. 16A-16D the multi-lumen extension 37E may be flexible and may be designed to bend upwards in such a manner that: (i) during insertion of the probe via the colonoscope working channel 14 the multi-lumen extension 37E is generally parallel to the probe longitudinal axis and\or the working channel main axis as shown in FIGS. 16A-16B, for example due to a force that the working channel potentially applies on a tip of the multi-lumen extension 37E; and (ii) once the tip of the multi-lumen extension 37E extends and protrudes from the working channel opening as shown in FIGS. 16C-16D, the multi-lumen extension 37E optionally bends upwards.

As rendered in FIG. 16B the loop 15B which passes over the multi-lumen extension may generate friction between the probe and the working channel wall 14. Such friction may damage the working channel inner wall surface. The loop 15B may be placed in an optional groove 37G which can potentially keep the loop 15B away from the working channel wall 14.

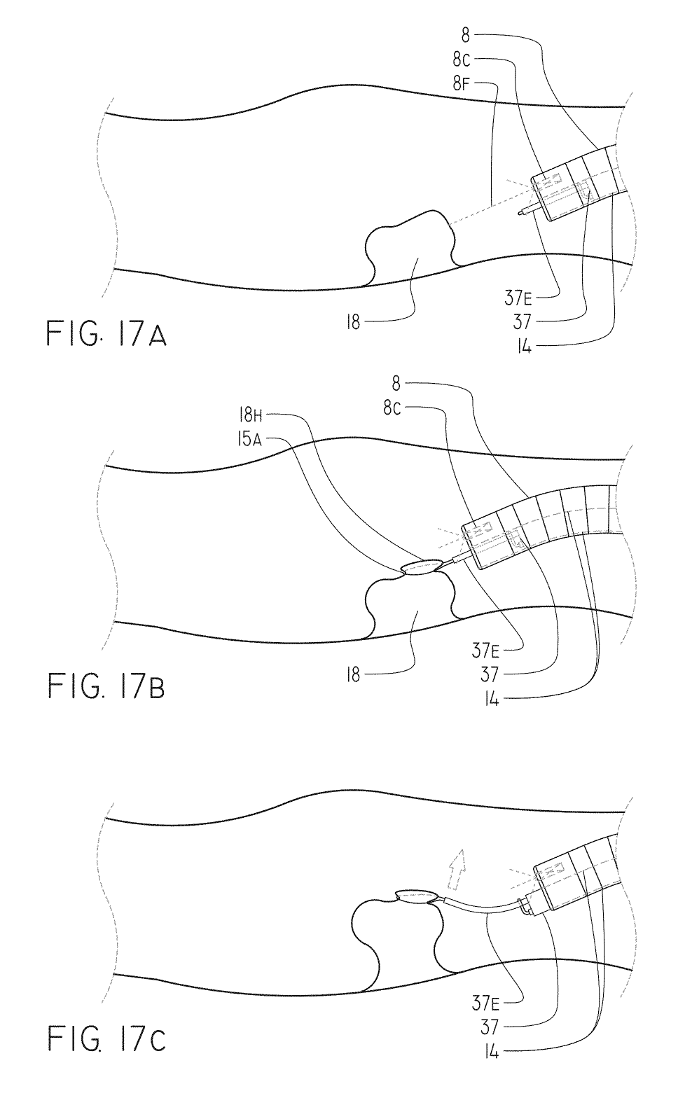

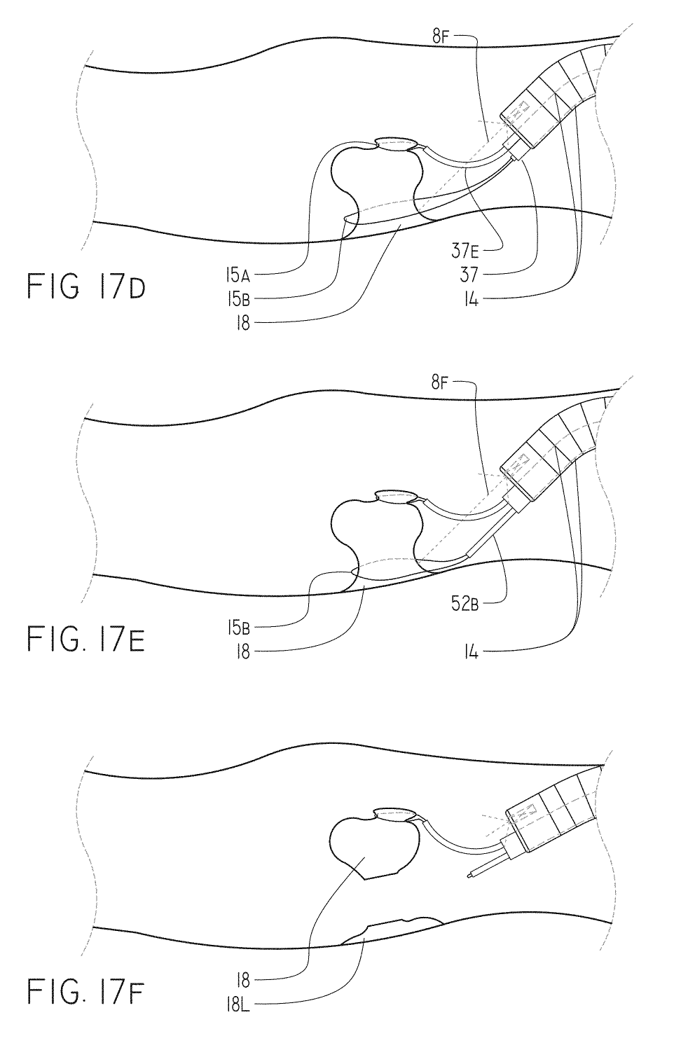

As rendered in FIGS. 17A-17F—the probe as described above may optionally be used to treat a polyp within a colon in a method described as “over the head”. The probe loops may be preloaded as described in FIGS. 16A-16B:

(i) Once a physician spots a polyp (for example a flat or sessile polyp) as rendered in FIG. 17A, the physician optionally stops the colonoscope movement in the colon, and optionally inserts a probe via a colonoscope working channel. During the insertion the multi-lumen extension is aligned within the working channel. In some embodiments the physician may choose to inject saline between the colon wall and the polyp prior to the polyp removal process.

The physician optionally pushes the probe until a tip of the multi-lumen extension extends from the working channel 14, and optionally until the tip can be viewed in the colonoscope camera 8C, as rendered in FIG. 17A. The multi-lumen extension tip may be viewed by the physician via the colonoscope camera similarly to viewing a standard snare. The physician optionally steers a tip of the colonoscope next to the polyp 18 and optionally loosens the first loop 15A enabling first loop 15A to open. The physician optionally steers the tip of the colonoscope until the first loop 15A has contact with a surface of the head of the polyp 18H, as rendered in FIG. 17A. The physician optionally closes the first loop 15A, optionally by manipulating an external probe handle. The first loop 15A grasps the polyp head 18A, or some other part of the polyp head, as rendered in FIG. 17B.

Field Of View (FOV): During the above operations the physician has view of the polyp, the polyp head and a tip of the first loop 15A, similar to a snare procedure. A center of the field of view is marked as 8F in FIG. 17A.

(ii) The physician optionally pushes the tip of the probe forward in the colon, and out of the working channel opening. Such forward movement may free the multi-lumen extension to bend upward and pull the polyp head 18H up and away from the colon wall, as rendered in FIG. 17C. Such an upward pull has a clinical value as described above. Such an upward pull potentially clears the FOV of the first loop 15A, and enables viewing a leg or a base of the polyp.

(iii) The physician optionally loosens the lower loop 15B, enabling the lower loop 15B to open, and optionally pushing the lower loop 15B to move in a linear manner over the upper loop 15A and into the colon, as rendered in FIG. 17D. When the loop 15B is open, as rendered in FIG. 17E, the physician optionally steers the colonoscope tip downward, and optionally closes the second loop 15B by pushing the over tube 52B forward and\or by pulling the second loop 15B backwards. Once the second loop 15B is closed on the base of the polyp the physician may optionally activate an electrosurgical generator, and treatment can optionally be commenced in a monopolar or bipolar setting.

FOV: During the above operation the physician potentially has a view of the polyp leg or the polyp base and the second loop 15B, as rendered in FIG. 17E. The second loop 15B is potentially centered in the field of view is marked as 8F and the physician potentially has an optimal view of a working area.

(iv) FIG. 17F shows the polyp after cutting away from the colon wall, and optionally still grasped by the first loop 15A.

Method of Removing a Polyp in an Accurate Manner

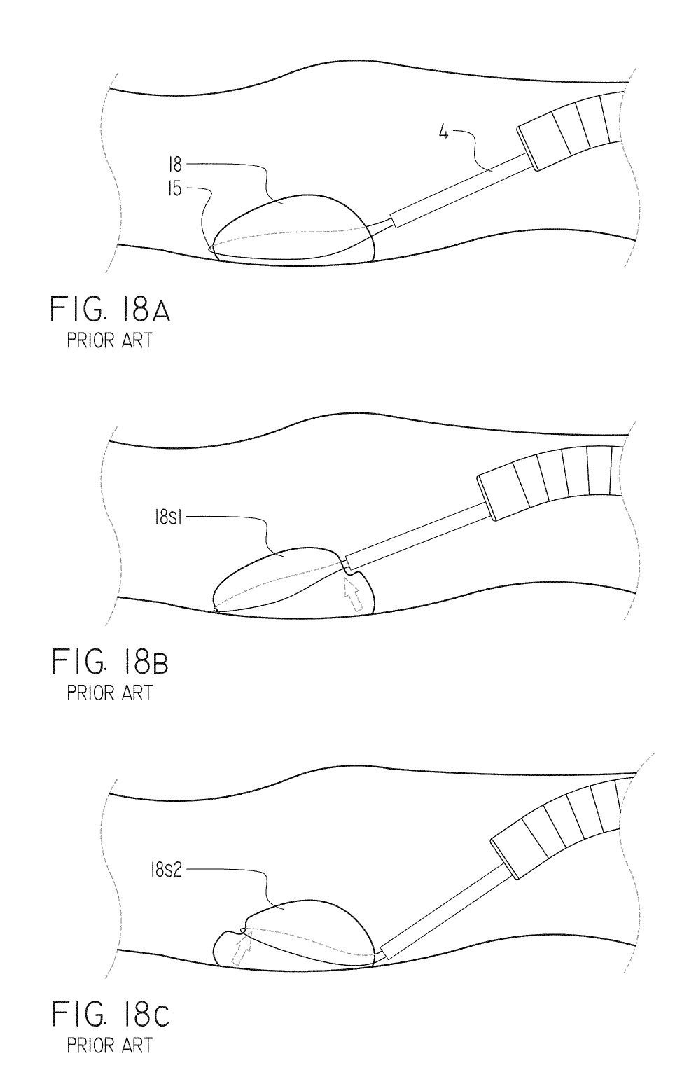

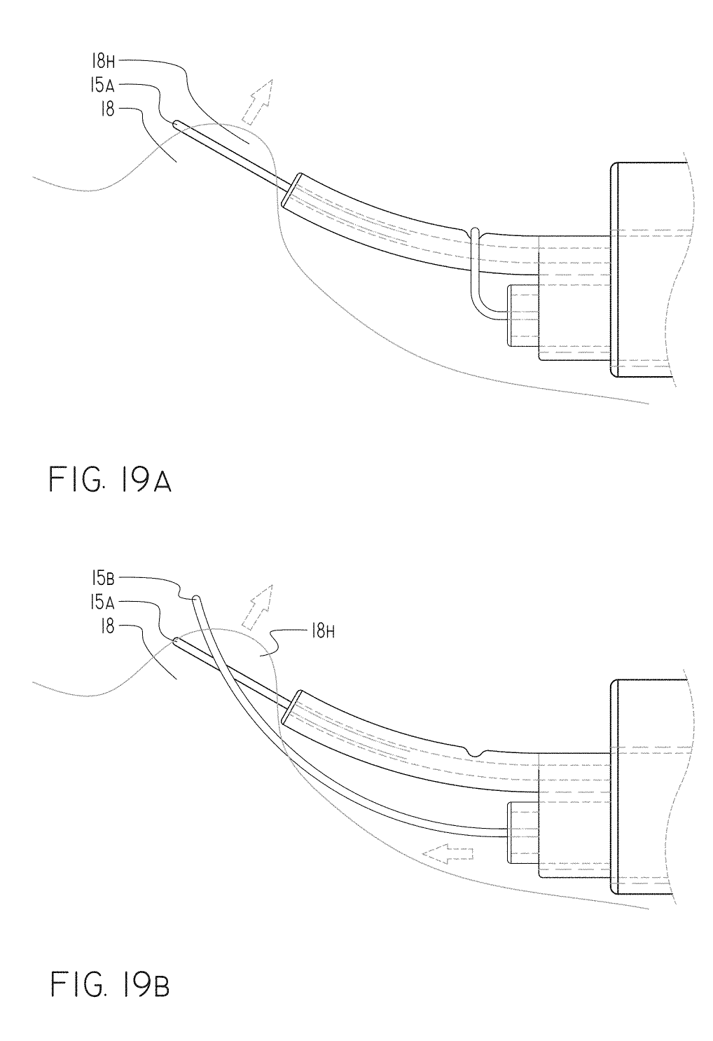

Reference is now made to FIGS. 18A-18C, which are simplified schematic illustrations of a snare and a polyp according to prior art.

As rendered in FIGS. 18A-18C—when removing a flat or sessile polyp using snares, a physician may find it hard to capture an entire polyp with a snare's loop. The physician optionally steers a probe's tip sideways or up-down by steering a colonoscope tip using external colonoscope controls. Once an open loop 15 is placed on the polyp 18 in an optimal manner, as rendered in FIG. 18A, the physician may optionally start closing and tightening the loop 15. The closing the loop 15 may result in (i) closing at an optimal location or; (ii) slippage of the tip (or the base) of the loop 15 and closing the loop 15 on the polyp segments 18S1 or 18S2 as rendered in FIGS. 18B-18C or; (iii) a complete failure to grab any part of the polyp by the loop 15.

Slippage of the loop may happen due to several reasons: the colon mucosa natural lubrication, a hump like polyp profile (unlike pedunculated polyps with a narrow neck and a wide head), movement of the colonoscope during the closing of the loop, movement of the probe during the closing of the loop, peristaltic movement of the colon, and other reasons.

FIG. 18A depicts a situation where the physician may start closing the loop 15 when the probe is in an optimal location, but due to the colon's mucosa natural lubrication, for example, the base of the loop may slide upward as shown in FIG. 18B.

FIG. 18C depicts a situation where the physician may try grabbing the polyp for a second time. The physician may steer the colonoscope down and push a tip of the probe downward to the colon wall. The base of the loop is then located in the right location on the polyp, but due to the flexible nature of the loop, the tip of the loop may move upwards. In such a case, once the loop 15 is closed, only a segment of the polyp 18S2 may be captured. To resolve such a scenario, the physician may use a very stiff loop which will not bend upward during such a process. The use of a stiff loop may potentially generate other problems, for example higher forces applied to the colon wall thus enlarging the risk of colon perforation.

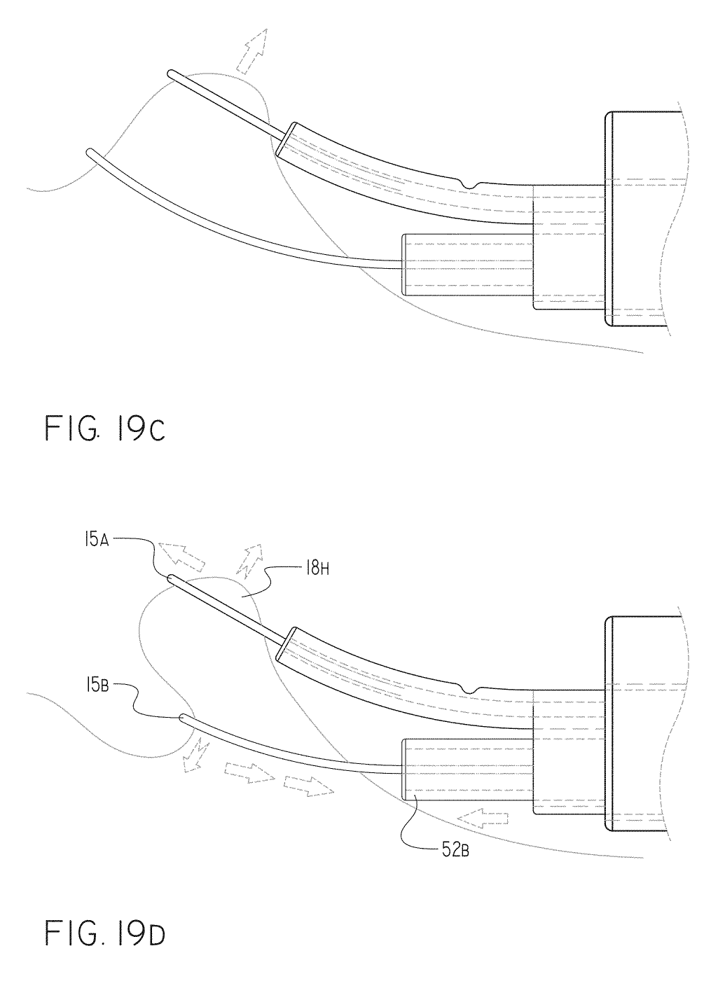

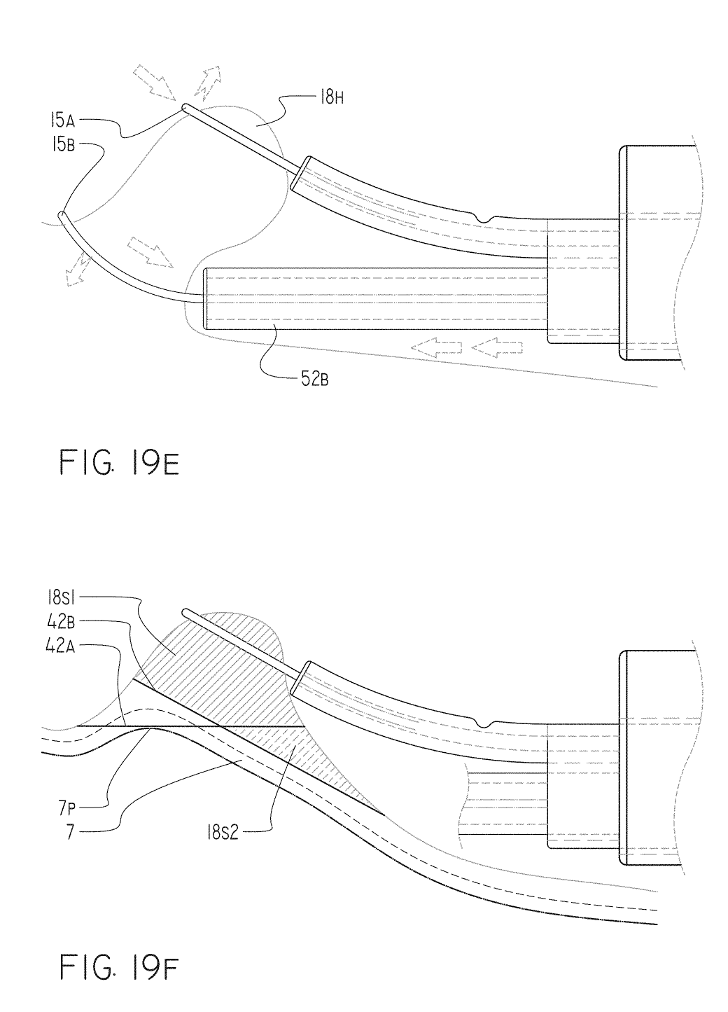

Reference is now made to FIGS. 19A-19F, which are simplified schematic illustrations of a probe according to an embodiment of the invention.

As rendered in FIGS. 19A-19F, the probe as described above is optionally used to treat a polyp within a colon in a method described as “over the head” with a high accuracy. FIG. 19A depicts the physician having grabbed a head of a polyp 18H with a first loop 15A, and elevated the polyp head up and away from the colon wall. FIG. 19B depicts the physician having deployed the second loop 15B by pushing the second loop 15B forward and expanding the second loop 15B. FIG. 19C depicts the physician having placed the two loops 15A 15B in position, and possibly starting to close the lower loop 15B prior to cutting the polyp.

The probe of FIGS. 19A-19F potentially enables the physician to choose a location at which the second loop 15B will be closed on the polyp. Two options are described below as examples of the way a physician may use such an accuracy feature.

FIG. 19D renders a manner wherein the physician optionally “holds the head of the polyp—and closes a loop on the proximal part of the polyp” (proximal relative to the colonoscope camera). The physician optionally operates the probe in such a manner that (i) the head of the polyp is pulled upward by the first loop 15A and the base of the polyp is pushed downward by the second loop 15B and; (ii) the first loop 15A also pushes the head of the polyp forward while the second loop 15B is pulls the base of the polyp backwards.

Such movement potentially forces the first loop 15A to close on the polyp head and on proximal tissue of the polyp.

FIG. 19E renders a manner in which the physician optionally “holds the head of the polyp—and closes a loop on a distal part of the polyp” (distal relative to the colonoscope camera). The physician optionally operates the probe in such a manner that (i) the head of the polyp is pulled upward by the first loop 15A and the base of the polyp is pushed downward by the second loop 15B and; (ii) the over-tube 52B is pushed forward while (iii) the second loop 15B is pulled, closing on the base of the polyp.

Such movement potentially forces the second loop 15B to close on the polyp and on distal polyp tissue.

Such a feature potentially enables the physician to control the location where the loops are closed and to gain accuracy in removal of the polyp tissue. Two overlapping scenarios are rendered in FIG. 19F as an example. If the physician closes the loop using a standard snare, the snare's loop will be closed “wherever it ends up” and when the physician uses the electrosurgical generator the result may end up cutting randomly e.g. 18s1 or 18s2 as shown in FIG. 18B-C. (a) If the physician cuts along cutting line 42A the cutting line 42A may be too close to the colon wall and colon wall perforation 7P may result. Moreover, the tissue marked as 18S1 will be removed and the tissue marked as 18S2 will not be cut away. Such tissue 18S2 may generate cancer, for example, in the future. (b) If the physician cuts along cutting line 42B the polyp tissue 18S1 and 18S2 are removed and the colon wall is kept safe.

A similarly accurate example embodiment may be performed by rotating the probe around its main axis. The physician may rotate the probe within the colonoscope working channel, generating movement of the first loop 15A and the second loop 15B. Such movement of the first loop 15A can potentially pull the polyp head right or left relative to the second loop 15B—potentially enabling the physician to close the second loop 15B on the right side or the left side of the polyp.

In some of the embodiments described herein with reference to various polyps and/or tissue, a polyp size as described with reference to FIG. 19 may be of 2-mm to 6 mm to 20 mm to 60 mm, although such embodiments potentially suit larger and smaller sizes.

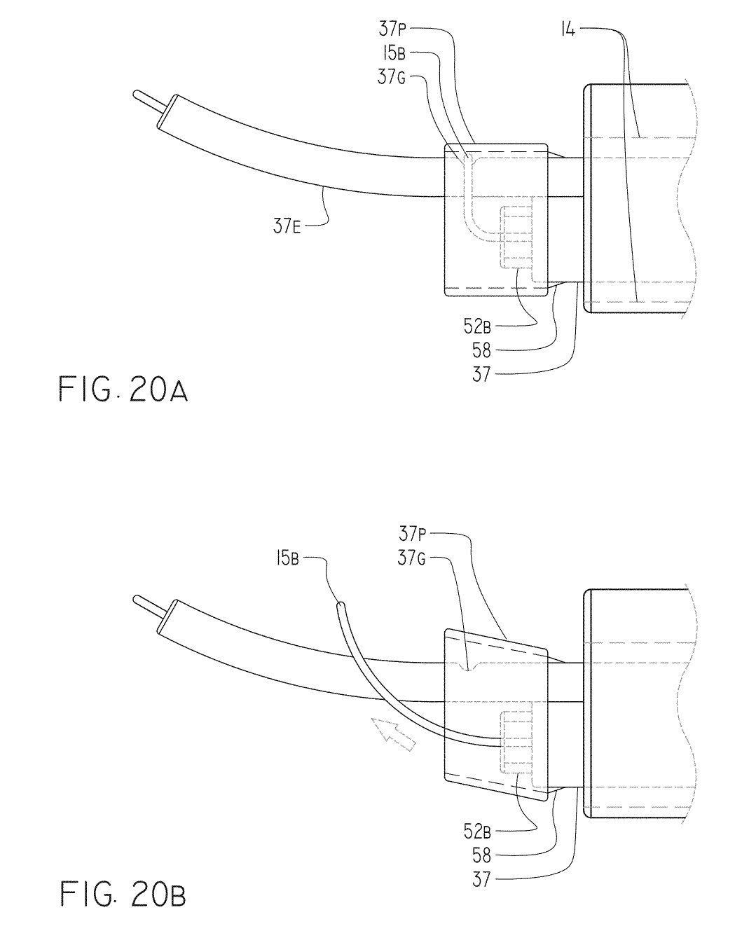

Reference is now made to FIGS. 20A-20B, which are simplified schematic illustrations of a probe with a protective cover according to an embodiment of the invention.

FIGS. 20A-20B show a probe including a protective shield 37P which potentially keeps the probe and the colonoscope working channel intact during the insertion of the probe via the working channel. A loop 15B may be passed over a multi-lumen extension 37E and may optionally be placed within a multi lumen groove 37G to keep the loop 15B far from an inner wall of the working channel 14. Such a groove potentially provides protection, and to enhance such protection a multi lumen protective cover 37P may optionally be added. Such a protective cover may optionally be flexible and may optionally be attached, for example glued 58, to the multi lumen 37 to keep the protective cover in place during an insertion phase. After the probe has been inserted in the colon via the working channel, as rendered in FIG. 20A, the physician may optionally push the loop 15B forward and the protective cover opening may deflect sideways, enabling the loop 15B to open and move forward as rendered in FIG. 20B.

Example Method of Removing a Polyp

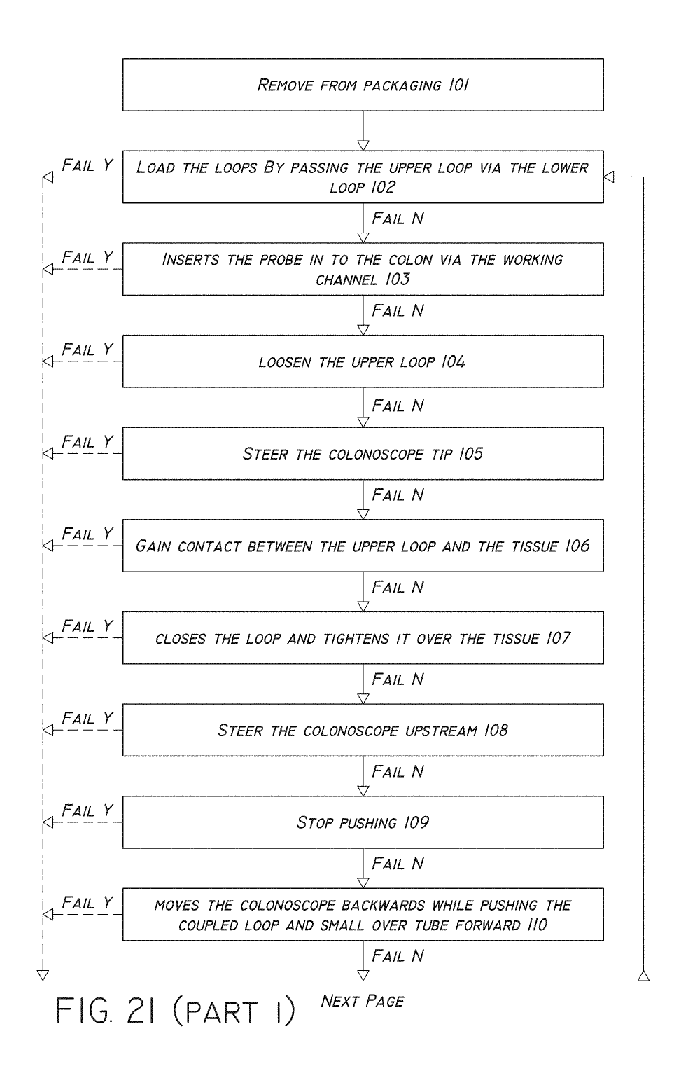

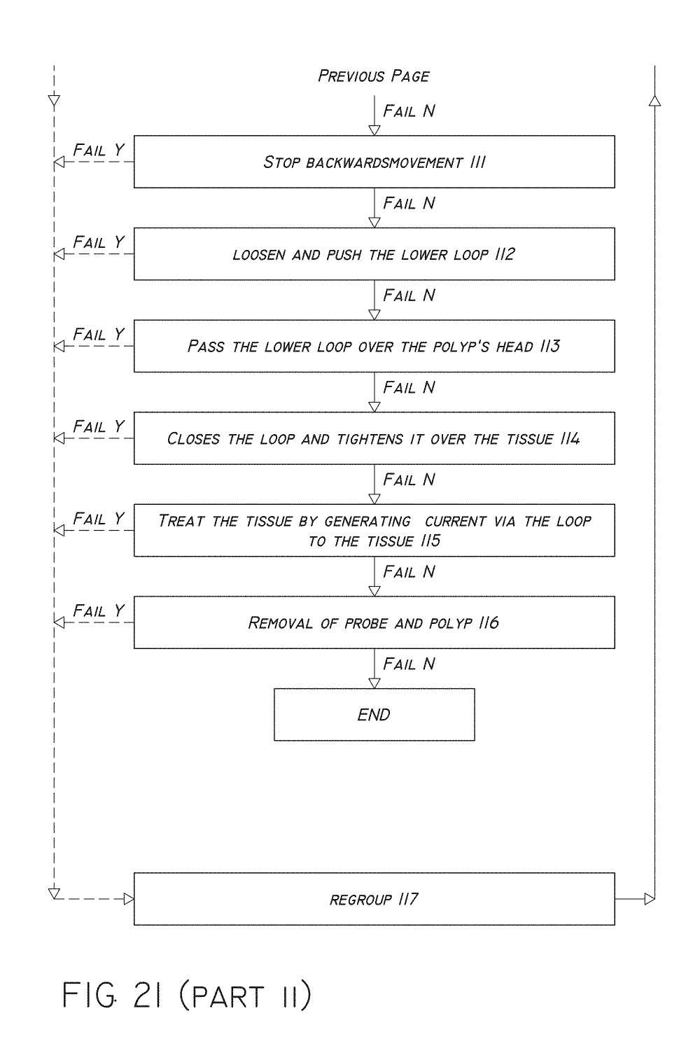

Reference is now made to FIG. 21, which is a simplified flow chart of a method for removing a polyp according to an example embodiment of the invention.

At 102, a physician optionally loads the loops prior to commencing treatment. In the exemplary embodiment depicted in FIG. 21, the physician optionally loads the probe by passing an upper loop through a lower loop. The physician may optionally use a probe's controls in order to loosen or tighten each loop during the loading process. Such loading activity may be done using the physician hands only or using a prefabricated jig.

In some embodiments loading the loops is performed during assembly and the physician does not perform 102.

After the loading process is done, at 103, the probe is optionally inserted via a colonoscope working channel and in to the colon.

At 104, optionally causing the probe's tip to protrude from the working channel, the physician may loosen the upper loop and steer the colonoscope tip (at 105) so that the upper loop achieves good contact with tissue to be treated (at 106). Such a tissue may be, by way of a non-limiting example, a part of a polyp head.

At 107, the physician optionally closes the upper loop and tightens the upper loop over the tissue until establishing a grasp of the tissue, as rendered, by way of a non-limiting example, in FIG. 14A. The closing and tightening process may be done in two manners (i) pushing a small over tube forward (ii) pulling the loop backwards in to the over tube. For ease of use the physician may optionally lock the upper loop and its over tube in such a manner that the loop be kept closed. Such a lock may optionally be accomplished by restricting linear movement between the loop and its over tube. Such a locking may enable other degrees of movement to the locked loop and over tube combination, for example; both loop and its over tube may move forward or backward together or rotate together. For ease of explanation such locked loop and over tube will be referred to as “upper coupled loop and small over tube”.

At 108, the physician optionally pushes and steers the colonoscope, for example upstream, with the probe's loop grasping the head of the polyp, optionally using the colonoscope optics to see that the polyp and tools are moved in a safe path and manner. During this movement the physician may push the upper loop and small over tube forward to gain additional movement of the polyp head.

At 109, the physician has pushed the polyp head all the way, and the physician ceases pushing, by way of a non-limiting example as rendered in FIG. 14B.

At 110, the physician moves the colonoscope, for example downstream or backwards, while pushing the upper coupled loop and small over tube forward via the colonoscope working channel. Such a movement potentially keeps the polyp head in its upstream location, but during such a movement the polyp head may move somewhat downstream or upstream without harm to the process. Such a movement can be performed as a (i) simultaneous movement by moving the colonoscope and the upper coupled loop and small over tube at a same time and by a same amount or (ii) segmental movement by moving the colonoscope a portion of the needed distance then moving the upper coupled loop and small over tube a portion of the needed distance, optionally moving the colonoscope again in an iterative manner.

At 111, the physician may optionally stop such movement, optionally once the physician gains (i) a view of the polyp leg or (ii) a view of a lumen of the colon with the polyp located upstream or (iii) a partial view of the polyp.

At 112, the physicians optionally loosens and/or pushes the lower loop, by way of a non-limiting example as rendered in FIG. 14C, until the lower loop reaches above the polyp head (at 113), by way of a non-limiting example as rendered in FIG. 14D. The physician may optionally steer the tip of the colonoscope or move the tip of the probe in order to facilitate such movement.

At 114, with the lower loop potentially passed over the polyp head; the physician optionally closes the lower loop. Such a closing and/or tightening process may be performed in two manners (i) pushing the lower over tube forward (ii) pulling the lower loop backwards in to the over tube. For ease of use the physician may lock the lower loop and its over tube as described above with reference to the upper loop. For ease of explanation such a locked loop and its over tube will be referred to as a “lower loop and small over tube”.

At 115, the physician may optionally treat the polyp. The treatment may be done for example in a (i) monopolar manner, where current flows from the lower loop to a grounding pad, or in a (ii) bipolar manner where current flows from the upper loop to the lower loop. The physician may treat the polyp, by way of a non-limiting example, as rendered in FIG. 14E, or, by way of another non-limiting example, release the upper loop and use the lower loop only during such treatment, as rendered in FIG. 14F.

It is possible that during the process described above the physician may fail to complete a stage due to difficulties, e.g. grasping the polyp head in a firm manner; passing the lower loop over the polyp head. If the physician fails to perform a task at a first attempt, the physician may stop and regroup for a new attempt (at 117).

Such a regroup may optionally require (i) a loosening of the lower loop and/or the upper loop from the tissue, if applicable, and (ii) a removal of the probe from the colonoscope working channel (iii) reloading the loops as described above and commencement of a new attempt as described above.

If the physician spots a second polyp, the physician may need to reload prior to treating the second polyp.

Example Method of Controlling Force of Grasping and/or Cutting

Once a physician grabs tissue with a grasping tool the physician may close an external handle controlling a grasping tool and lock handle of the grasping tool. The physician may lock the grasping tool to free his/her hands to operate a second handle manipulating a cutting tool. However, even if the external handle locks the grasping tool in an optimal manner, the grasping tool may eventually open slightly due to changes (for example movement of the tissue, the colon wall or breathing cycle of a patient). Moreover, there may be long cables between the external handle and the inner grasping tool—in a colonoscopy setting such a length may be of 2200-2400 mm. Such a long path may allow opening of the grasping tool over time.

In some embodiments the grasping tool is optionally connected to the external handle by a mechanism, such as a wire which includes a spring for maintaining tension.

Such a spring potentially enables the physician to lock the grasping tool and ensure that the grip of the grasping tool on tissue will remain firm during the polyp removal procedure. Such a spring may be designed to apply an optimal force on tissue being held by the grasping tool.

Such a spring may be included within the grasping tool, or coupled externally to the grasping tool, for example along a wire connecting the grasping tool to the external handle.

Such a mechanism for maintaining tension may optionally be included in a cutting tool. The physician may tension the mechanism before cutting, and release the tensioning mechanism to pull on the cutting tool, assisting the physician to maintain pressure on the cutting tool, optionally maintaining an approximately constant cutting pressure. By maintaining approximately constant pressure the cutting tool may be prevented from potentially cutting too fast, without allowing heating time for coagulation, or preventing from potentially cutting too slow, causing heat damage and maybe even drying tissue lowering conductance, as described above.

In some embodiments a restraining mechanism or a compensation mechanism is optionally placed between a tool such as a loop on a distal end of a probe and a handle manipulated by a physician. Such a restraining mechanism may be, by way of some non-limiting examples, a spring or an elastic band. The placement of such a spring between the loop and the handle potentially assists in maintaining tension on the tool, potentially keeping tissue held with a grasping tool or in touch with a cutting tool, removing the need for a physician to pay specific attention to continuously maintain tension on a control handle.

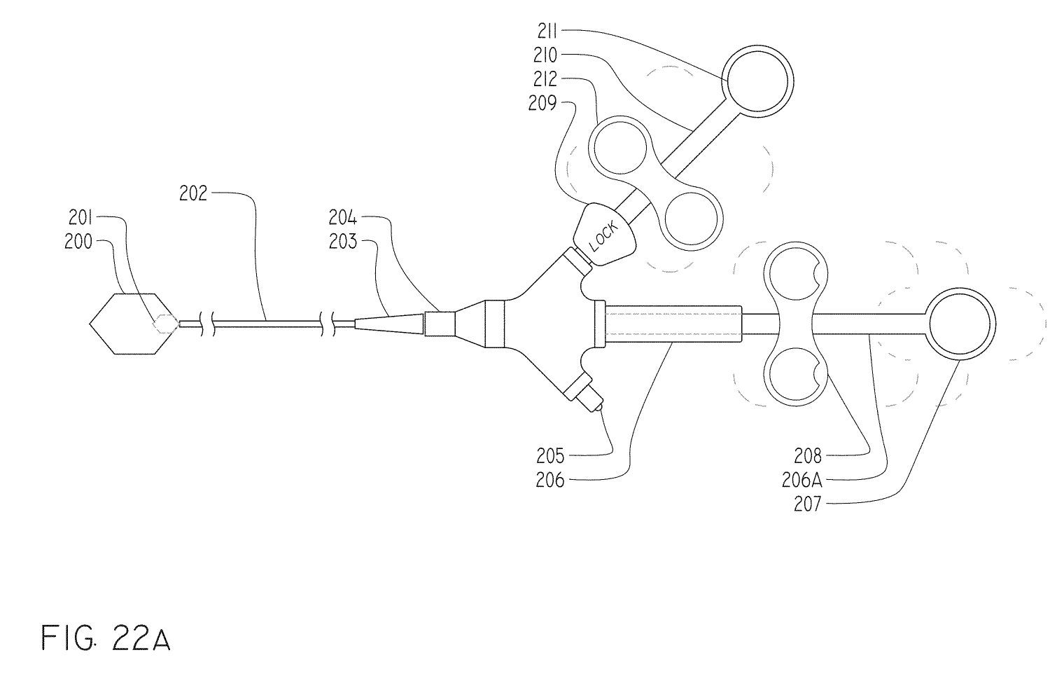

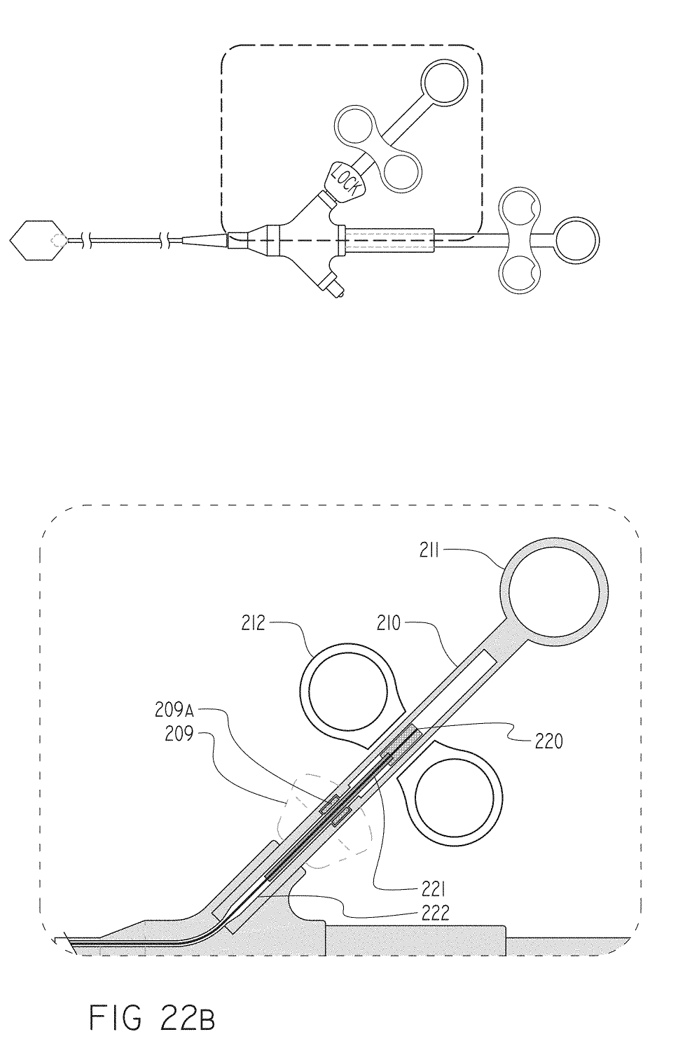

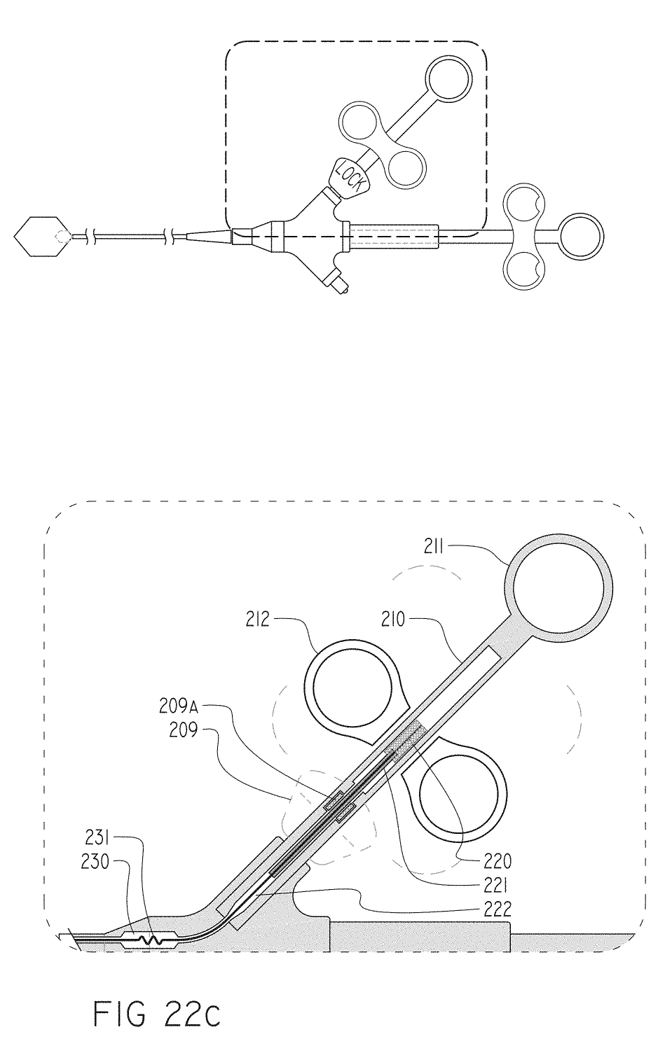

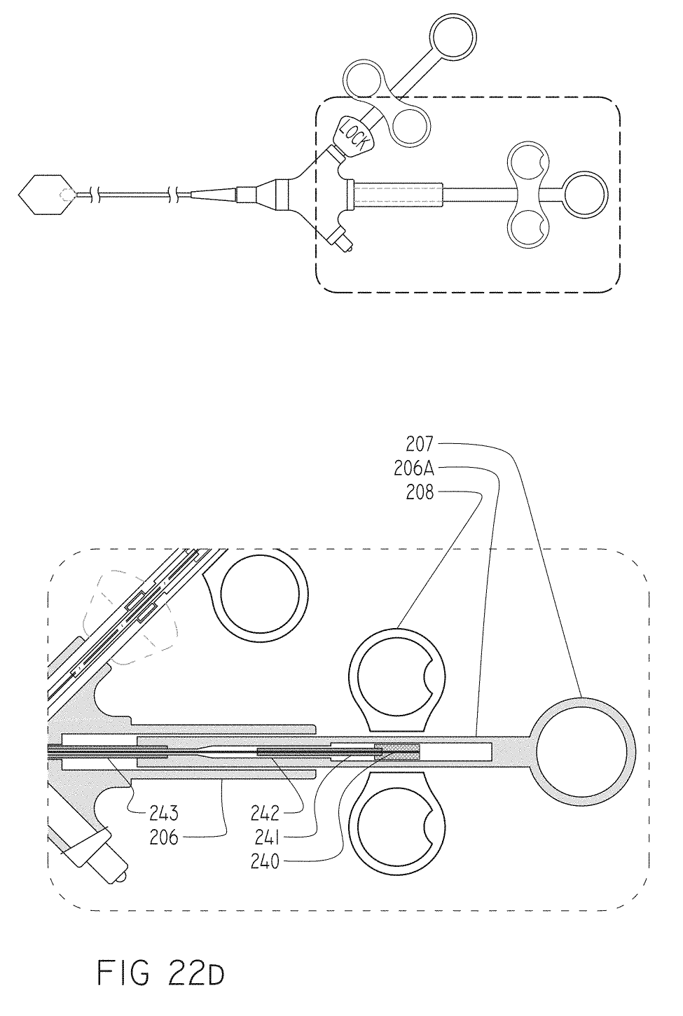

Reference is now made to FIGS. 22A-22D, which are simplified schematic illustrations of a probe according to an example embodiment of the invention.

As rendered in FIG. 22A, a device including a grasping tool 201 and a cutting tool 200. In the example embodiment depicted in FIG. 22A, the grasping tool and the cutting tool include loops. Both the grasping tool loop 201 and the cutting tool loop 200 are optionally manipulated by cables which pass via a shaft 202. The shaft 202 is optionally connected to an external handle housing 204, optionally via a strain release section 203. The cutting loop 200 and the grasping loop 201 are optionally activated via an external handle. The grasping loop cable is optionally manipulated by a handle 212 and the cutting loop cable is optionally manipulated by a handle 208.

The grasping loop 201 may be manipulated in one degree of freedom, opening and closing the grasping loop by optionally moving the handle 212 relative to another handle 211. Such a degree of freedom may be locked in place using a locking mechanism 209.

The cutting loop 200 is optionally connected electrically to an electrical connector 205. The cutting loop 200 may optionally be manipulated in several degree of freedom by: (i) opening and closing the cutting loop 200 by moving the handle 208 relative to a handle 207; (ii) moving the handle 207, which is optionally connected to a rotor 206A in a linear manner relative to the external handle housing 204; (iii) moving the handle 207, which is connected to the rotor 206A in a rotational manner relative to the external handle housing 204.

As rendered in FIG. 22B, the grasping tool handle is zoomed with a cross section showing internals of the handle mechanism. The grasping loop (not shown) 201 connected via a cable 220 which passes via the device shaft (not shown). The cable 220 is connected to the handle 212. The handle 212 slides in a linear manner within a stator 210. The cable 212 is housed within a tube 221, which tube 221 may optionally be locked by the locking mechanism 209 and optional locking teeth 209A. The tube 221 optionally moves in a linear manner within a cavity 222 within the external handle housing 204.

As rendered in FIG. 22C, the grasping tool handle is zoomed with a cross section showing internals of the handle mechanism. The grasping loop (not shown) 201 is connected via a cable 220 which passes via the device shaft (not shown). The cable 220 is optionally connected to the handle 212 via a spring 231 which may move within a spring cavity 230. The handle 212 slides in a linear manner within the stator 210. The cable 212 is optionally housed within a tube 221. The tube 221 may optionally be locked by a locking mechanism 209 optionally by locking teeth 209A. The spring 231 may optionally elongate due to movement of the handle 212 when the grasping loop is closed on the polyp. Such a spring potentially keeps the polyp tissue in grasp even if the polyp changes in size during a polyp removal procedure, and/or even if the physician moves the colono scope during the procedure.

As rendered in FIG. 22D, the cutting loop 200 handle is zoomed with a cross section showing internals of such a handle mechanism. The cutting loop (not shown) 200 is optionally connected via a cable 240 which passes via the device shaft (not shown). The cable 240 is optionally connected to a handle 208. The handle 208 optionally slides in a linear manner within a rotor 206A. The cable 240 is optionally housed within a tube 241. The tube 241 optionally moves in a linear manner within a rotor cavity 242. Such a movement potentially enables opening and closing of the cutting loop 200. The handle 208, the rotor 206A and the handle 207 can move together in a linear and/or rotational manner within the cavity 206 located in the external handle housing 204. Such a movement can potentially generate linear or rotational movement of the cutting loop within the colon.

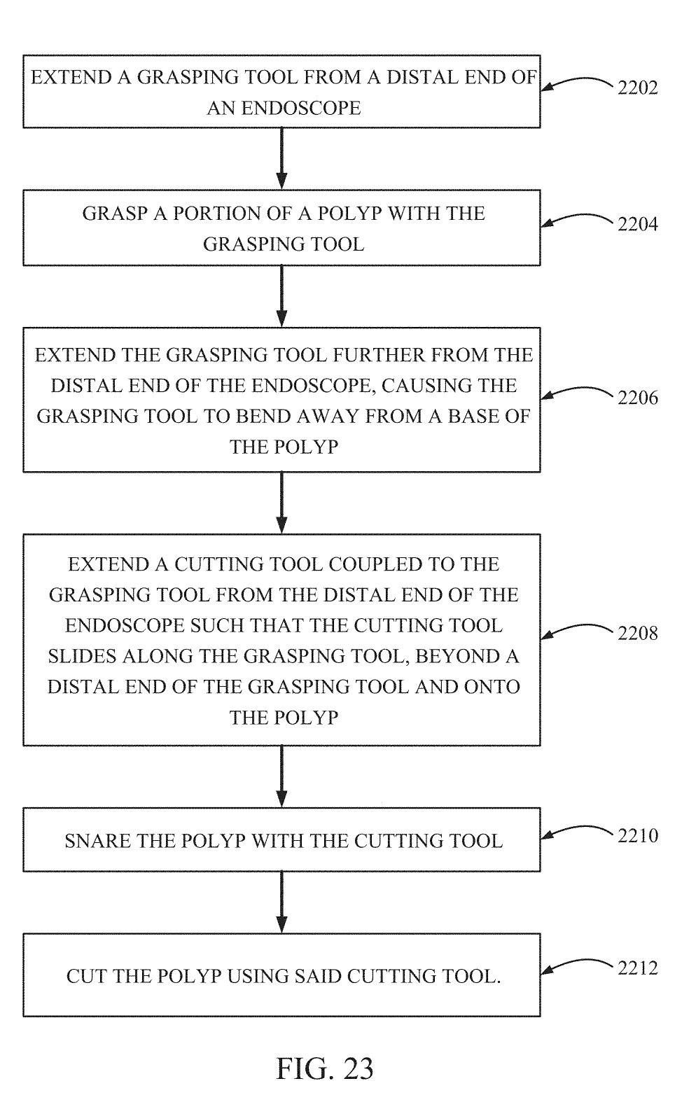

Reference is now made to FIG. 23, which is a simplified flow chart of a method for removing a polyp according to an example embodiment of the invention.

The method of FIG. 23 includes:

extending a grasping tool from a distal end of an endoscope (2202);

grasping a portion of a polyp with the grasping tool (2204);

extending the grasping tool further from the distal end of the endoscope, thereby causing the grasping tool to bend away from a base of the polyp (2206);

extending a cutting tool coupled to the grasping tool from the distal end of the endoscope such that the cutting tool slides along the grasping tool, beyond a distal end of the grasping tool and onto the polyp (2208);

snaring the polyp with the cutting tool (2210); and

cutting the polyp using the cutting tool (2212).

In some embodiments the cutting includes mechanical cutting.

In some embodiments the cutting includes electrically cutting.

In some embodiments the extending the grasping tool further from the distal end of the endoscope to cause the grasping tool to bend includes extending a sheath enveloping the grasping tool further from the distal end of the endoscope to cause the grasping tool sheath to bend.

In some embodiments the cut polyp is retrieved from a patient's body using the grasping tool, optionally by virtue of the grasping tool still grasping the cut polyp after the cutting.

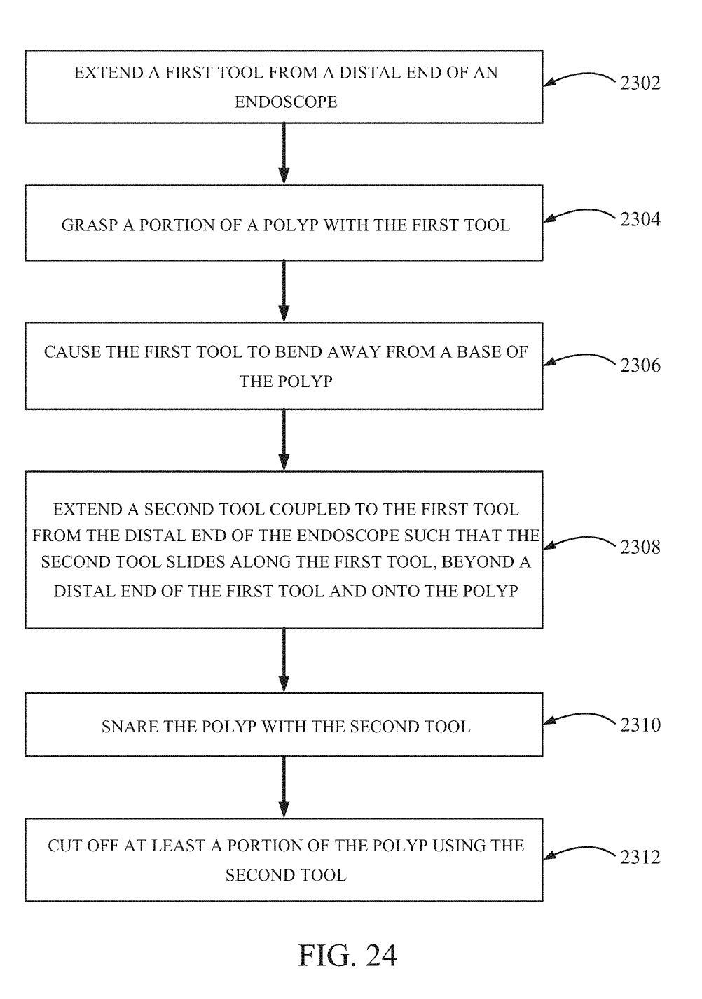

Reference is now made to FIG. 24, which is a simplified flow chart of a method for removing a polyp according to an example embodiment of the invention.

The method of FIG. 24 includes:

extending a first tool from a distal end of an endo scope (2302);

grasping a portion of a polyp with the first tool (2304);

causing the first tool to bend away from a base of the polyp (2306);

extending a second tool coupled to the first tool from the distal end of the endoscope such that the second tool slides along the first tool, beyond a distal end of the first tool and onto the polyp (2308);

snaring the polyp with the second tool (2310); and

electrically cutting off at least a portion of the polyp using the second tool (2312).

In some embodiments the cutting includes mechanical cutting.

In some embodiments the cutting includes electrically cutting.

In some embodiments the cut polyp is retrieved from a patient's body using the grasping tool, optionally by virtue of the grasping tool still grasping the cut polyp after the cutting.

Some of the disclosure described herein is directed to descriptions of exemplary embodiments intended for the use in polyp removal within the colon, but it is to be understood that systems similarly constructed and similarly used are expected to be useful in other bodily organs, for example in the urinary tract or other body cavity, and the length and diameters of the probe and other working parts may optionally be adapted to the different locations they may be used in a body.

Although descriptions provided are largely couched in terms of exemplary embodiments designed for use in the intestinal tract, the inventive embodiments described herein are not necessarily limited to those exemplary embodiments but should be understood to include any tissue treatment system for the use anywhere inside or outside the body which comprises the elements described and/or claimed herein.

It is expected that during the life of a patent maturing from this application many relevant endoscopes will be developed, and the scope of the term “endoscope” or “colonoscope” is intended to include all such new technologies a priori. Similarly, it is expected that during the life of a patent maturing from this application many relevant probes for treating the GI tract will be developed, and the scope of the term “probe”, where appropriate in context, is intended to include all such new technologies a priori.

Embodiment of the invention; certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Citation or identification of any reference in this application shall not be construed as an admission that such reference is available as prior art to the present invention.

To the extent that section headings are used, they should not be construed as necessarily limiting.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A device for cutting polyps via an endoscope lumen, comprising:
an over tube arranged to pass through an endoscope, the over tube comprising:
(1) a grasping tool arranged to be controlled from a proximal end of said endoscope and to controllably extend from a distal end of said endoscope; and
(2) a cutting tool arranged to pass through said endoscope, said cutting tool arranged to be controlled from said proximal end of said endoscope and to controllably extend from said distal end of said endoscope,
in which:
at least one of said grasping tool and said cutting tool is configured to bend away from the other one by comprising a pre-bent element configured to bend away when extended from said endoscope, and biased to a flat configuration when inside said endoscope; and
said cutting tool is coupled to said grasping tool.

2. A device according to claim 1, in which said cutting tool is coupled to said grasping tool while said grasping tool and said cutting tool are inside said over tube, and said cutting tool is arranged to slide along said grasping tool when pushed toward a distal end of said grasping tool.

3. A device according to claim 1, in which said cutting tool comprises a loop coupled to said grasping tool while said grasping tool and said loop are inside said over tube, and said loop is arranged to slide over said grasping tool when pushed toward a distal end of said grasping tool.

4. A device according to claim 1, in which:
said device further comprises a grasping tool sheath enveloping said grasping tool;
said cutting tool envelopes said grasping tool sheath while said grasping tool and said cutting tool are inside said over tube; and
said cutting tool is arranged to slide along said grasping tool sheath and said grasping tool when pushed toward a distal end of said grasping tool.

5. A device according to claim 1, and further comprising:
a tensioning mechanism attached to said grasping tool, operational to maintain grasping force on tissue by maintaining tension between a distal end of said grasping tool and a manipulating mechanism at a proximal end of said grasping tool.

6-10. (canceled)

11. A device according to claim 1, in which said device further comprises a cutting tool sheath enveloping a portion of said cutting tool, and said cutting tool is configured to controllable extend from said cutting tool sheath.

12. (canceled)

13. A device according to claim 1, in which said over tube comprises a separator between a first lumen through said over tube enveloping said grasping tool and a second lumen through said over tube enveloping said cutting tool.

14. A device according to claim 1, in which said endo scope is one of a group consisting of:
an arthroscope;
an amnioscope;
a laryngoscope;
a colposcope;
a hysteroscope;
a laparoscope;
a sygmoidoscope;
a hysteroscope;
a gastroscope;
a colonoscope; and
a cystoscope.

15. (canceled)

16. A device according to claim 4, in which said grasping tool sheath is configured to bend away from said cutting tool when said cutting tool is extended beyond said grasping tool and said grasping tool sheath is extended out of said over tube.

17. A device according to claim 4, in which said grasping tool sheath further comprises at least one shape memory insert configured to bend away from said cutting tool when said grasping tool sheath is extended out of said over tube.

18. A device according to claim 17, in which said at least one shape memory insert extends from a distal end of said grasping tool sheath to a proximal end of said over tube.

19. A device according to claim 4, in which said grasping tool sheath further comprises at least a spring insert configured to bend away from said cutting tool when said grasping tool sheath is extended out of said endoscope.

20. A device according to claim 1, in which said cutting tool comprises a snare.

21. A device according to claim 1, in which said cutting tool comprises a loop.

22. (canceled)

23. A device according to claim 1, in which said grasping tool is one of a group consisting of:
a loop;
a snare;
a grasper;
a biopsy forceps;
a tweezers-like grasper; and
a harpoon.

24-28. (canceled)

29. A surgical method comprising:
extending a grasping tool from a distal end of an endoscope;
grasping a portion of a polyp with said grasping tool;
extending said grasping tool further from said distal end of said endoscope, thereby causing said grasping tool to bend away from a base of said polyp;
extending a cutting tool coupled to said grasping tool from said distal end of said endoscope such that said cutting tool slides along said grasping tool, beyond a distal end of said grasping tool and onto said polyp;
snaring said polyp with said cutting tool; and
cutting said polyp using said cutting tool.

30. (canceled)

31. A method according to claim 29 in which said extending said grasping tool further from said distal end of said endoscope causing said grasping tool to bend away from a base of said polyp comprises extending a sheath enveloping said grasping tool further from said distal end of said endoscope causing said grasping tool sheath to bend away from a base of said polyp.

32. A method according to claim 29 and further comprising:
using a camera at a distal end of said endoscope to observe said grasping said portion of a polyp with said grasping tool; and
following said extending said grasping tool further from said distal end of said endoscope causing said grasping tool to bend away from a base of said polyp, using said camera to observe said snaring said polyp with said cutting tool.

33. A method according to claim 32 and further comprising using said camera to observe said cutting said polyp using said cutting tool.

34-36. (canceled)

37. A surgical method comprising:
extending a first tool from a distal end of an endoscope;
grasping a portion of a polyp with said first tool;
causing said first tool to bend away from a base of said polyp;
extending a second tool coupled to said first tool from said distal end of said endoscope such that said second tool slides along said first tool, beyond a distal end of said first tool and onto said polyp;
snaring said polyp with said second tool; and
cutting off at least a portion of said polyp using said second tool.

38-41. (canceled)
